(19) United States
(12) Patent Application Publication
(10) Pub. No.: US 2026/0013979 A1
(43) Pub. Date: Jan. 15, 2026

(54) STENT DEVICE

(71) Applicant: OLYMPUS CORPORATION, Tokyo (JP)

(72) Inventors: Mamoru HASEGAWA, Kamiina-gun (JP); Shun NOGUCHI, Machida-shi (JP); Toshiaki HAYASHI, Hachioji-shi (JP)

(21) Appl. No.: 19/327,707

(22) Filed: Sep. 12, 2025

Related U.S. Application Data
(63) Continuation of application No. PCT/JP2023/014069, filed on Apr. 5, 2023.

(51) Int. Cl. A61F 2/04

(52) U.S. Cl. CPC A61F 2/04; A61F 2002/041; A61F 2002/045; A61F 2230/0069; A61F 2230/0091

(57) ABSTRACT

A stent device includes a stent formed in a tubular shape by weaving wires and a cover covering at least a part of the stent. The cover is partially bonded to parts rather than a whole surface of the stent.

### CROSS-REFERENCE TO RELATED APPLICATIONS

Priority is claimed on PCT/JP2023/014069, filed on Apr. 5, 2023, the entire content of which is incorporated herein by reference.

### FIELD

The present disclosure relates to a stent device.

### BACKGROUND

A procedure of detaining a stent in a stenosis or obstruction (hereinafter referred to as a “stenosis or the like”) occurring in a digestive tract or the like and expanding the stenosis or the like is known. A stent delivery system is used to detain a stent in a stenosis or the like. The stent delivery system is inserted into a treatment tool channel of an endoscope and delivers a stent to a stenosis or the like.

A stent device (a covered stent) in which a stent is covered with a thin film (a cover) is used. The stent device covered with a film (a cover) can prevent cells or blood from infiltrating into a stent in a stenosis or the like.

In Published Japanese Translation No. 2017-522091 of the PCT International Publication (hereinafter referred to as Patent Document 1), a covered stent which can easily maintain a bent state is described. The covered stent described in Patent Document 1 is formed by causing an inner tape and an outer tape to pass through a rhomboidal space portion between wires and bonding them together.

### SUMMARY

A stent device includes a stent formed in a tubular shape by weaving wires and a cover covering at least a part of the stent, wherein the cover is partially bonded to parts rather than the whole surface of the stent.

### BRIEF DESCRIPTION OF THE DRAWINGS

FIG. 1 is a diagram illustrating a whole configuration of an endoscope system according to a first embodiment.

FIG. 2 is a diagram illustrating a stent device of the endoscope system.

FIG. 3 is a diagram illustrating a hooking portion of the stent device.

FIG. 4 is a diagram illustrating a modified example of a cover of the stent device.

FIG. 5 is a diagram illustrating another modified example of the cover of the stent device.

FIG. 6 is a diagram illustrating another modified example of the cover of the stent device.

FIG. 7 is a diagram illustrating another modified example of the cover of the stent device.

FIG. 8 is a diagram illustrating another modified example of the cover of the stent device.

FIG. 9 is a diagram illustrating another modified example of the cover of the stent device.

FIG. 10 is a diagram illustrating a stent device according to a second embodiment.

FIG. 11 is a diagram illustrating a modified example of the stent device.

FIG. 12 is a diagram illustrating a manufacturing method of the stent device.

FIG. 13 is a diagram illustrating a manufacturing method of the stent device.

FIG. 14 is a diagram illustrating a manufacturing method of the stent device.

FIG. 15 is a diagram illustrating another modified example of the stent device.

FIG. 16 is a diagram illustrating a manufacturing method of another modified example of the stent device.

FIG. 17 is a diagram illustrating a manufacturing method of the modified example.

FIG. 18 is a diagram illustrating a manufacturing method of the modified example.

FIG. 19 is a diagram illustrating a manufacturing method of another modified example of the stent device.

FIG. 20 is a diagram illustrating a manufacturing method of the modified example.

FIG. 21 is a diagram illustrating a manufacturing method of the modified example.

FIG. 22 is a diagram illustrating a manufacturing method of the modified example.

FIG. 23 is a diagram illustrating another modified example of the stent device.

FIG. 24 is a diagram illustrating another modified example of the stent device.

FIG. 25 is a diagram illustrating a stent device according to a third embodiment.

FIG. 26 is a diagram illustrating a manufacturing method of the stent device.

FIG. 27 is a diagram illustrating a manufacturing method of the stent device.

FIG. 28 is a diagram illustrating a modified example of the stent device.

FIG. 29 is a diagram illustrating a stent device according to a fourth embodiment.

FIG. 30 is a diagram illustrating a manufacturing method of the stent device.

FIG. 31 is a diagram illustrating a manufacturing method of the stent device.

FIG. 32 is a diagram illustrating a manufacturing method of the stent device.

FIG. 33 is a diagram illustrating a modified example of the stent device.

FIG. 34 is a diagram illustrating another modified example of the stent device.

FIG. 35 is a diagram illustrating another modified example of the stent device.

FIG. 36 is a diagram illustrating a stent device according to a fifth embodiment.

FIG. 37 is a diagram illustrating a manufacturing method of the stent device.

FIG. 38 is a diagram illustrating a manufacturing method of the stent device.

FIG. 39 is a diagram illustrating a manufacturing method of the stent device.

FIG. 40 is a diagram illustrating a manufacturing method of the stent device.

FIG. 41 is a diagram illustrating a manufacturing method of the stent device.

FIG. 42 is a diagram illustrating operations and effects of the stent device.

FIG. 43 is a diagram illustrating operations and effects of the stent device.

FIG. 44 is a diagram illustrating a modified example of the stent device.

FIG. 45 is a diagram illustrating a manufacturing method of the modified example.

FIG. 46 is a diagram illustrating a manufacturing method of the modified example.

FIG. 47 is a diagram illustrating a manufacturing method of the modified example.

FIG. 48 is a diagram illustrating a stent device according to a sixth embodiment.

FIG. 49 is a diagram illustrating a manufacturing method of the stent device.

FIG. 50 is a diagram illustrating a manufacturing method of the stent device.

FIG. 51 is a diagram illustrating a manufacturing method of the stent device.

FIG. 52 is a diagram illustrating an example of combinations of the embodiments.

FIG. 53 is a diagram illustrating another example of a combination of the embodiments.

FIG. 54 is a diagram illustrating another example of a combination of the embodiments.

FIG. 55 is a diagram illustrating another example of a combination of the embodiments.

FIG. 56 is a diagram illustrating another example of a combination of the embodiments.

FIG. 57 is a diagram illustrating another example of a combination of the embodiments.

FIG. 58 is a diagram illustrating another example of a combination of the embodiments.

FIG. 59 is a diagram illustrating another example of a combination of the embodiments.

FIG. 60 is a diagram illustrating another example of a combination of the embodiments.

FIG. 61 is a diagram illustrating another example of a combination of the embodiments.

### DETAILED DESCRIPTION

In the covered stent described in Patent Document 1 or the like, a bonding portion between the tape located outside and the tape located inside may hinder movement of a wire. When movement of a wire is hindered by the bonding portion, the covered stent is less likely to be bent.

One aspect of the present disclosure can provide a stent device (a covered stent) that is more likely to be bent even when a stent is covered with a cover.

The stent device according to the present disclosure is more likely to be bent even when a stent is covered with a cover.

### First Embodiment

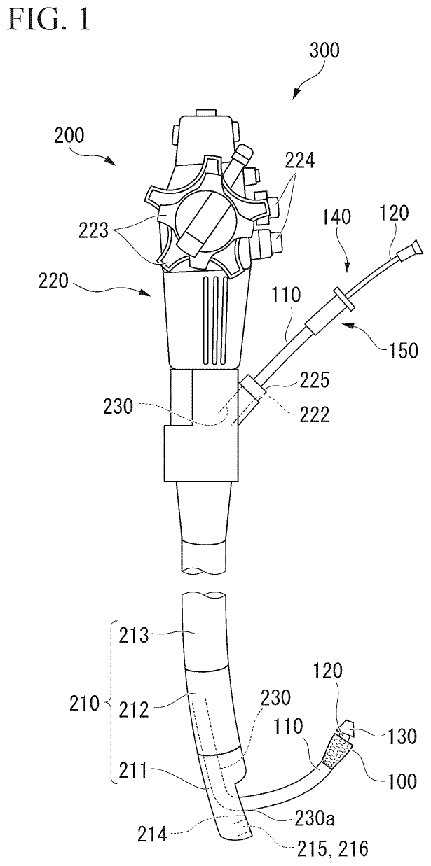

An endoscope system 300 including a stent device 100 according to a first embodiment will be described below with reference to FIGS. 1 to 9. FIG. 1 is a diagram illustrating the entire configuration of the endoscope system 300.

### [Endoscope System 300]

The endoscope system 300 includes an endoscope 200 and a stent delivery system 150 which is inserted into a channel of the endoscope 200.

### [Endoscope 200]

The endoscope 200 is a known side-view flexible endoscope and includes a longitudinal insertion unit 210 and an operation unit 220 that is provided at a proximal end of the insertion unit 210. The endoscope 200 may be a direct-view flexible endoscope.

The insertion unit 210 includes a distal hard portion 211 provided at a distal end, a bendable bending portion 212 provided on a proximal side of the distal hard portion 211, and a flexible tubular portion 213 provided on a proximal side of the bending portion 212. An imaging unit 216 including a light guide 215 and a CCD is provided on a side surface of the distal hard portion 211 in a state in which the imaging unit is exposed to the outside.

A treatment tool channel 230 into which an endoscopic treatment tool such as the stent delivery system 150 is inserted is formed in the insertion unit 210. A distal end 230a of the treatment tool channel 230 is open on the side surface of the distal hard portion 211. A proximal end of the treatment tool channel 230 extends to the operation unit 220.

An erecting base 214 is provided in the distal hard portion 211 of the treatment tool channel 230. A proximal end of the erecting base 214 is rotatably supported by the distal hard portion 211. An erecting base operating wire (not illustrated) fixed to a distal end of the erecting base 214 extends to the proximal side through the inside of the insertion unit 210.

The bending portion 212 is configured to be bendable in a vertical direction or a lateral direction. A distal end of an operating wire is fixed to a distal end of the bending portion 212. The operating wire extends to the operation unit 220 through the inside of the insertion unit 210.

A knob 223 operating the operating wire and a switch 224 operating the imaging unit 216 or the like is provided on the proximal side of the operation unit 220. A user can bend the bending portion 212 in a desired direction by operating the knob 223.

A forceps port 222 communicating with the treatment tool channel 230 is provided on the distal side of the operation unit 220. A user can insert an endoscopic treatment tool such as the stent delivery system 150 from the forceps port 222. A forceps plug 225 is attached to the forceps port 222 in order to prevent leakage of a body fluid.

### [Stent Delivery System 150]

The stent delivery system 150 is formed to be thin and long as a whole and includes a stent device 100, an outer tubular member 160, an inner tubular member 170, and an operation unit 140.

The outer tubular member 160 is formed in a tubular shape out of a resin or the like and has flexibility. The outer tubular member 160 can be inserted into the treatment tool channel 230 of the endoscope 200.

The inner tubular member 170 has an outer radius smaller than an inner radius of the outer tubular member 160 and can communicate with an internal space (a lumen) of the outer tubular member 160. The inner tubular member 170 is formed of a resin or the like and has flexibility. A tip 180 with an outer radius larger than an outer radius of the outer tubular member 160 is provided at a distal end of the inner tubular member 170.

The stent device 100 is accommodated in a distal end portion of the stent delivery system 150 as illustrated in FIG. 1. The stent device 100 is accommodated in a space between the inner tubular member 170 and the outer tubular member 160 in a state in which the inner tubular member 170 is inserted thereinto and a radius thereof has decreased.

The operation unit 140 is connected to the proximal sides of the outer tubular member 160 and the inner tubular member 170 and is configured to move the outer tubular member 160 in a longitudinal direction relative to the inner tubular member 170. An operator can expose the accommodated stent device 100 and thus detain the stent device 100 by moving the outer tubular member 160 relative to the inner tubular member 170 by operating the operation unit. When a stent is exposed, the operator can also re-accommodate (re-capture) the stent device 100 in the space between the inner tubular member 170 and the outer tubular member 160 by moving the outer tubular member 160 relative to the inner tubular member 170 in the reverse direction.

### [Stent Device 100]

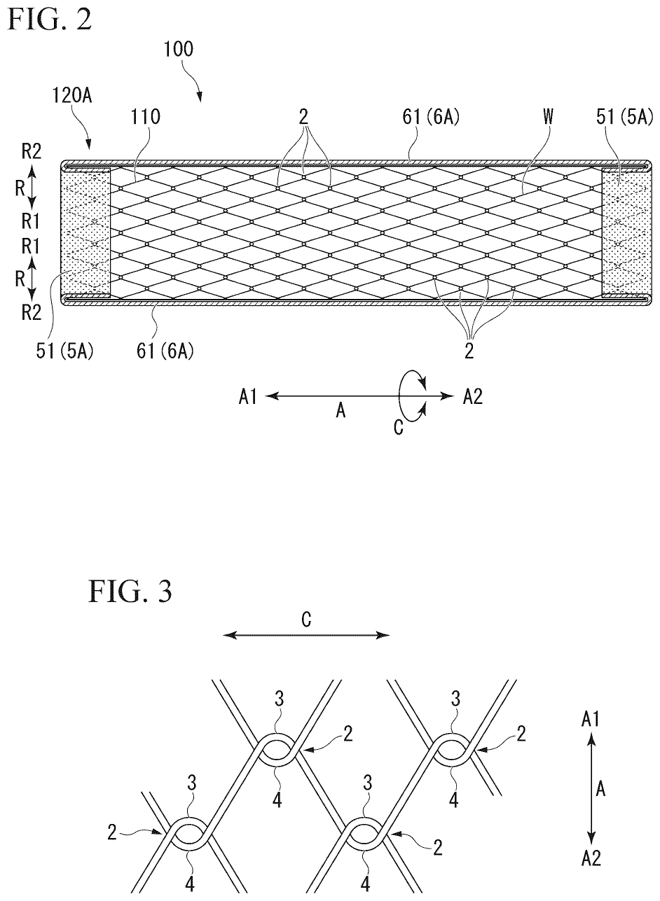

FIG. 2 is a diagram illustrating the stent device 100.

The stent device 100 is detained in a lumen of a digestive system such as a biliary duct, a gullet, a duodenum, a small intestine, or a large intestine and is mainly used to extend and maintain the lumen. The stent device 100 includes a stent 110 and a cover 120A. The stent device 100 is a covered stent in which the stent 110 formed by weaving wires is covered with the cover 120A.

In the following description, one side in a longitudinal direction (axial direction) A of the stent device 100 is referred to as a “first direction A1,” and the other side in the longitudinal direction A of the stent device 100 is referred to as a “second direction A2.”

### [Stent 110]

The stent (strut) 110 is formed by weaving wires W and has a cylindrical shape. The stent 110 is formed in a cylindrical tube shape having meshes in a circumferential surface thereof out of wires W extending obliquely with respect to a circumferential direction C while repeatedly performing bending. The stent 110 includes a plurality of hooked portions 2.

FIG. 3 is a diagram illustrating the hooked portions 2.

Each hooked portion (entwined portion) 2 is formed by causing a mountain bent portion 3 and a valley bent portion 4 to cross each other. The mountain bent portion (mountain) 3 is a convex portion which is formed to be convex to the first direction A1 of the longitudinal direction A by folding and bending a wire W extending obliquely with respect to the circumferential direction C. The valley bent portion (valley) 4 is a convex portion which is formed to be convex to the second direction A2 (a concave portion which is formed to be concave to the first direction A1) of the longitudinal direction A by folding and bending a wire W extending obliquely with respect to the circumferential direction. Since the mountain bent portion 3 and the valley bent portion 4 cross each other in a hook shape in the hooked portion 2, the mountain bent portion 3 and the valley bent portion 4 are connected to be non-separable and relatively movable.

Neighboring hooked portions 2 in the circumferential direction C are disposed substantially at the same position in the longitudinal direction A. Neighboring hooked portions 2 in the longitudinal direction A are disposed substantially at the same position in the circumferential direction C.

As illustrated in FIG. 2, the cover 120A is formed by overlapping an inner cover 5A disposed on the inside R1 in a radial direction R of the stent 110 and an outer cover 6A disposed on the outside R2 in the radial direction R of the stent 110.

The inner cover 5A is disposed partially in both end portions of the stent 110. Specifically, the inner cover 5A includes an end cylindrical portion 51 disposed in both end portions of the stent 110.

The end cylindrical portion 51 is formed in a cylindrical shape of which a side surface extends continuously in the circumferential direction C of the stent 110.

The outer cover 6A is disposed on the entire surface of the stent 110. Specifically, the outer cover 6A includes a body portion 61 disposed in both end portions and a central portion (a portion interposed between both end portions) of the stent 110. The outer cover 6A may include a folded portion 62 which is folded to the inside R1 of the stent 110.

The body portion 61 is formed in a cylindrical shape of which a side surface extends continuously in the circumferential direction C of the stent 110. The body portion 61 is provided on the entire outer circumferential surface of the stent 110 and extends from an end on the first direction A1 side to an end on the second direction A2 side of the stent 110.

The inner cover 5A and the outer cover 6A are not bonded on the entire surface of the stent 110 but are partially bonded. In both end portions of the stent 110, the end cylindrical portion 51 of the inner cover 5A is bonded to the entire surface of the body portion 61 of the outer cover 6A. When the outer cover 6A includes the folded portion 62, the end cylindrical portion 51 may be bonded to the folded portion 62.

The stent device 100 including the cover 120A include no bonding points 7 at which the inner cover 5A and the outer cover 6A are bonded in a central portion of the longitudinal direction A. Accordingly, the stent device 100 including the cover 120A has a smaller force resisting to bending (a smaller axial force) and is likely to be bent according to an internal organ shape.

### <Cover 120A1>

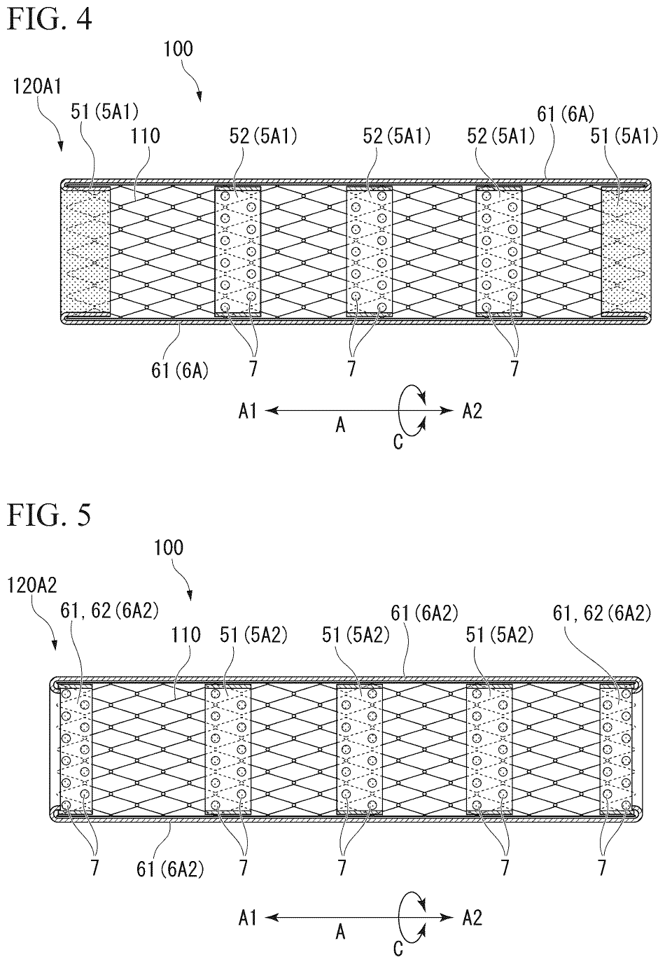

FIG. 4 is a diagram illustrating a cover 120A1 which is a modified example of the cover 120A.

The cover 120A1 is formed by overlapping an inner cover 5A1 disposed on the inside R1 in the radial direction R of the stent 110 and an outer cover 6A disposed on the outside R2 in the radial direction R of the stent 110.

The inner cover 5A1 is partially disposed in both end portions and a central portion of the stent 110. Specifically, the inner cover 5A includes an end cylindrical portion 51 disposed in both end portions of the stent 110 and a cylindrical portion 52 disposed in the central portion of the stent 110.

The cylindrical portion 52 is formed in a cylindrical shape of which a side surface extends continuously in the circumferential direction C of the stent 110. A plurality of cylindrical portions 52 are arranged at intervals in the longitudinal direction A.

The outer cover 6A is disposed on the entire surface of the stent 110. Specifically, the outer cover 6A includes a body portion 61 disposed in both end portions and the central portion of the stent 110.

In both end portions of the stent 110, the end cylindrical portion 51 of the inner cover 5A1 is bonded to the body portion 61 of the outer cover 6A on the entire surface thereof. In the central portion of the stent 110, the cylindrical portion 52 of the inner cover 5A1 is bonded to the body portion 61 of the outer cover 6A with a space (hereinafter referred to as a “cell”) surrounded with wires W interposed therebetween at points. The points at which the outer cover 6A and the inner cover 5A1 are bonded are referred to as “bonding points 7.”

The bonding points 7 between the cylindrical portions 52 of the inner cover 5A1 and the outer cover 6A are arranged in two lines in the circumferential direction C in FIG. 4, but the arrangement of the bonding points 7 are not limited thereto. The bonding points 7 may be arranged in three or more lines in the circumferential direction C.

The cover 120A1 may have a partial shape in which the end cylindrical portion 51 of the inner cover 5A1 is not disposed but only the outer cover 6A is disposed in both end portions of the stent 110.

The stent device 100 including the cover 120A1 has a smaller number of bonding points 7 at which the inner cover 5A1 and the outer cover 6A are bonded in the central portion in the longitudinal direction A. Accordingly, the stent device 100 including the cover 120A1 has a smaller force resisting to bending (a smaller axial force) and is more likely to be bent according to an internal organ shape.

Particularly, when the size of the stent device 100 is large, there is a likelihood that the magnitude of an expansion force will increase and cover separation or cover breakage will occur at the time of release and re-capturing of the stent device 100. In the stent device 100 including the cover 120A1, since the inner cover 5A1 bonded to the outer cover 6A is partially arranged in the central portion, it is possible to enhance a bonding force of the cover 120A1 through the bonding points 7 while maintaining axial force characteristics (characteristics less likely to resist to bending).

### <Cover 120A2>

FIG. 5 is a diagram illustrating a cover 120A2 which is a modified example of the cover 120A.

The cover 120A2 is formed by overlapping an inner cover 5A2 disposed on the inside R1 in the radial direction R of the stent 110 and an outer cover 6A2 disposed on the outside R2 in the radial direction R of the stent 110.

The inner cover 5A2 is partially disposed in the central portion of the stent 110. Specifically, the inner cover 5A2 includes a cylindrical portion 52 disposed in the central portion of the stent 110. The inner cover 5A2 does not include the end cylindrical portion 51.

The outer cover 6A2 is disposed on the entire surface of the stent 110. Specifically, the outer cover 6A2 includes a body portion 61 disposed in both end portions and the central portion and a folded portion 62 folded to the inside R1 of the stent 110 in both end portions.

In both end portions of the stent 110, the folded portion 62 of the outer cover 6A2 and the body portion 61 are bonded at points with a cell interposed therebetween. In the central portion of the stent 110, the cylindrical portion 52 of the inner cover 5A2 is bonded to the body portion 61 of the outer cover 6A2 with a cell interposed therebetween at points.

The stent device 100 including the cover 120A2 has a smaller number of bonding points 7 at which the inner cover 5A2 and the outer cover 6A2 are bonded in the central portion in the longitudinal direction A. Accordingly, the stent device 100 including the cover 120A2 has a smaller force resisting to bending (a smaller axial force) and is more likely to be bent according to an internal organ shape.

### <Cover 120A3>

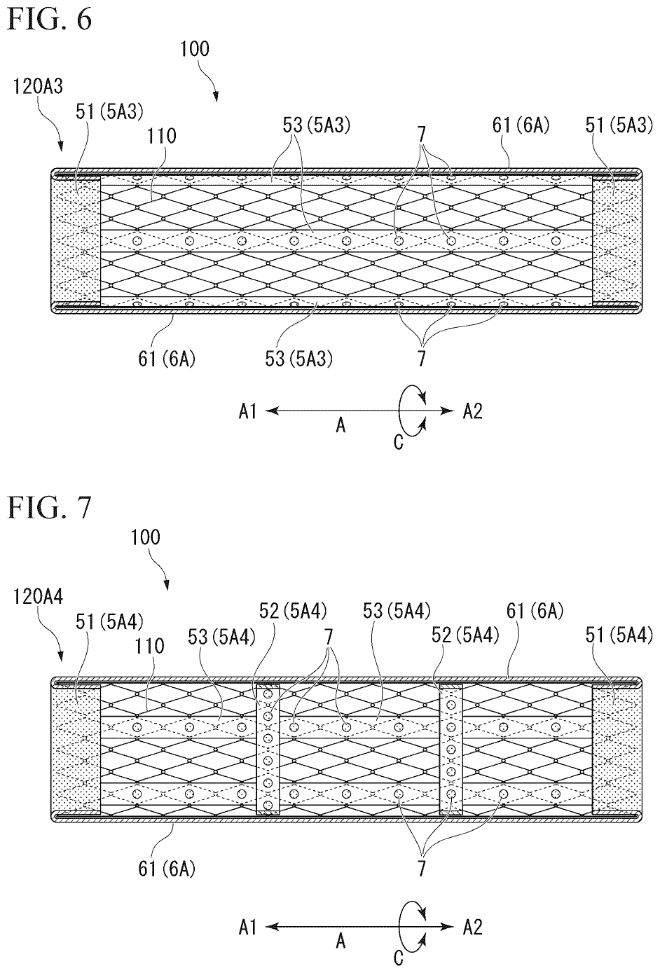

FIG. 6 is a diagram illustrating a cover 120A3 which is a modified example of the cover 120A.

The cover 120A3 is formed by overlapping an inner cover 5A3 disposed on the inside R1 in the radial direction R of the stent 110 and an outer cover 6A disposed on the outside R2 in the radial direction R of the stent 110.

The inner cover 5A3 is partially disposed in both end portions and the central portion of the stent 110. Specifically, the inner cover 5A3 includes an end cylindrical portion 51 disposed in both end portions of the stent 110 and a linear portion 53 disposed in the central portion of the stent 110.

The linear portion 53 is a linear member extending in the longitudinal direction A. A plurality of linear portions 53 are arranged at intervals in the circumferential direction C. The linear portions 53 are connected to the end cylindrical portion 51 in both end portions of the stent 110.

The outer cover 6A is disposed on the entire surface of the stent 110. Specifically, the outer cover 6A2 includes a body portion 61 disposed in both end portions and the central portion of the stent 110.

In both end portions of the stent 110, the end cylindrical portion 51 of the inner cover 5A3 is bonded to the body portion 61 of the outer cover 6A on the entire surface. In the central portion of the stent 110, the linear portions 53 of the inner cover 5A3 is bonded to the body portion 61 of the outer cover 6A with a cell interposed therebetween at points.

The stent device 100 including the cover 120A3 has a smaller number of bonding points 7 at which the inner cover 5A3 and the outer cover 6A are bonded in the central portion in the longitudinal direction A. Accordingly, the stent device 100 including the cover 120A3 has a smaller force resisting to bending (a smaller axial force) and is more likely to be bent according to an internal organ shape.

### <Cover 120A4>

FIG. 7 is a diagram illustrating a cover 120A4 which is a modified example of the cover 120A.

The cover 120A4 is formed by overlapping an inner cover 5A4 disposed on the inside R1 in the radial direction R of the stent 110 and an outer cover 6A disposed on the outside R2 in the radial direction R of the stent 110.

The inner cover 5A4 is partially disposed in both end portions and the central portion of the stent 110. Specifically, the inner cover 5A4 includes an end cylindrical portion 51 disposed in both end portions of the stent 110, a cylindrical portion 52 disposed in the central portion of the stent 110, and a linear portion 53 disposed in the central portion of the stent 110. The cylindrical portions 52 and the linear portions 53 cross each other. The linear portions 53 are connected to the end cylindrical portion 51 in both end portions of the stent 110.

The outer cover 6A is disposed on the entire surface of the stent 110. Specifically, the outer cover 6A includes a body portion 61 disposed in both end portions and the central portion of the stent 110.

In both end portions of the stent 110, the end cylindrical portion 51 of the inner cover 5A4 is bonded to the body portion 61 of the outer cover 6A on the entire surface. In the central portion of the stent 110, the cylindrical portions 52 and the linear portions 53 of the inner cover 5A4 are bonded to the body portion 61 of the outer cover 6A with a cell interposed therebetween at points.

The stent device 100 including the cover 120A4 has a smaller number of bonding points 7 at which the inner cover 5A4 and the outer cover 6A are bonded in the central portion in the longitudinal direction A. Accordingly, the stent device 100 including the cover 120A4 has a smaller force resisting to bending (a smaller axial force) and is more likely to be bent according to an internal organ shape.

### <Cover 120A5>

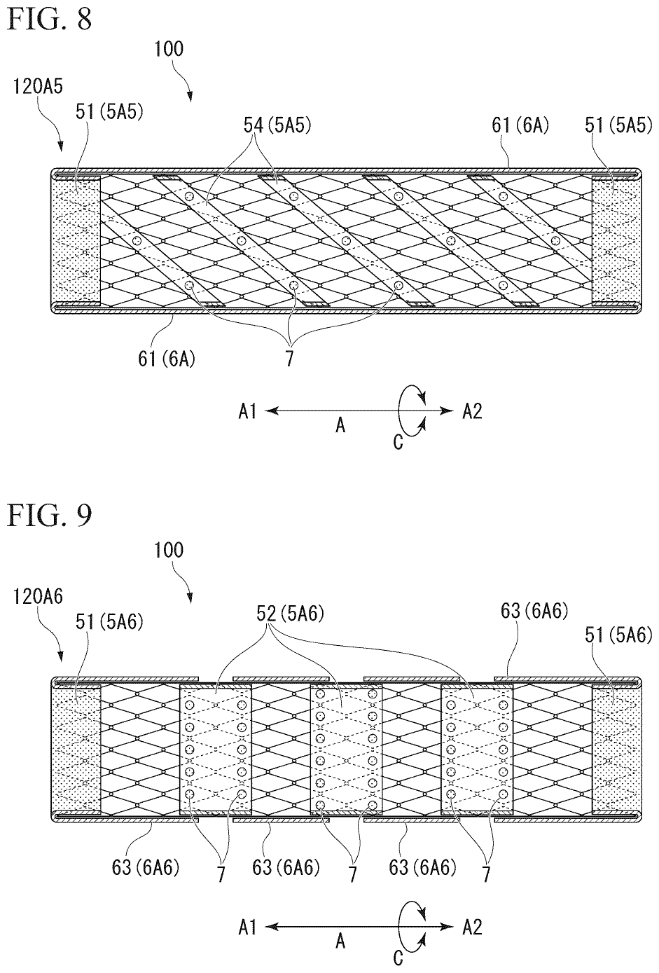

FIG. 8 is a diagram illustrating a cover 120A5 which is a modified example of the cover 120A.

The cover 120A5 is formed by overlapping an inner cover 5A5 disposed on the inside R1 in the radial direction R of the stent 110 and an outer cover 6A disposed on the outside R2 in the radial direction R of the stent 110.

The inner cover 5A5 is partially disposed in both end portions and the central portion of the stent 110. Specifically, the inner cover 5A5 includes an end cylindrical portion 51 disposed in both end portions of the stent 110 and a spiral portion 54 disposed in the central portion of the stent 110.

The spiral portion 54 is a linear member extending in a spiral shape in the longitudinal direction A. The spiral portion 54 is connected to the end cylindrical portion 51 in both end portions of the stent 110.

The outer cover 6A is disposed on the entire surface of the stent 110. Specifically, the outer cover 6A includes a body portion 61 disposed in both end portions and the central portion of the stent 110.

In both end portions of the stent 110, the end cylindrical portion 51 of the inner cover 5A4 is bonded to the body portion 61 of the outer cover 6A on the entire surface thereof. In the central portion of the stent 110, the spiral portions 54 of the inner cover 5A4 are bonded to the body portion 61 of the outer cover 6A with cells interposed therebetween at points.

The bonding points 7 between the spiral portions 54 of the inner cover 5A5 and the outer cover 6A are arranged in one line in FIG. 8, but the arrangement of the bonding points 7 is not limited thereto. The bonding points 7 may be arranged in two or more lines.

The cover 120A5 may have a partial shape in which the end cylindrical portion 51 of the inner cover 5A5 is not disposed but only the outer cover 6A is disposed in both end portions of the stent 110.

The stent device 100 including the cover 120A5 has a smaller number of bonding points 7 at which the inner cover 5A5 and the outer cover 6A are bonded in the central portion in the longitudinal direction A. Accordingly, the stent device 100 including the cover 120A5 has a smaller force resisting to bending (a smaller axial force) and is more likely to be bent according to an internal organ shape.

### <Cover 120A6>

FIG. 9 is a diagram illustrating a cover 120A6 which is a modified example of the cover 120A.

The cover 120A6 is formed by overlapping an inner cover 5A6 disposed on the inside R1 in the radial direction R of the stent 110 and an outer cover 6A6 disposed on the outside R2 in the radial direction R of the stent 110.

The inner cover 5A6 is partially disposed in both end portions and the central portion of the stent 110. Specifically, the inner cover 5A6 includes an end cylindrical portion 51 disposed in both end portions of the stent 110 and a cylindrical portion 52 disposed in the central portion of the stent 110.

The outer cover 6A6 is partially disposed in both end portions and the central portion of the stent 110. Specifically, the outer cover 6A6 includes cylindrical portions 63 disposed in both end portions and the central portion of the stent 110.

The cylindrical portions 63 are formed in a cylindrical shape of which a side surface extends continuously in the circumferential direction C of the stent 110. A plurality of cylindrical portions 63 are arranged at intervals in the longitudinal direction A. The cylindrical portions 52 of the inner cover 5A6 and the cylindrical portions 63 of the outer cover 6A6 are arranged alternately in the longitudinal direction A.

In both end portions of the stent 110, the end cylindrical portions 51 of the inner cover 5A6 are bonded to the cylindrical portions 63 of the outer cover 6A6 on the entire surface thereof. In the central portion of the stent 110, the cylindrical portions 52 of the inner cover 5A6 and the cylindrical portions 63 of the outer cover 6A6 are bonded with cells interposed therebetween at points.

The stent device 100 including the cover 120A6 has a smaller number of bonding points 7 at which the inner cover 5A6 and the outer cover 6A6 are bonded in the central portion in the longitudinal direction A. Accordingly, the stent device 100 including the cover 120A6 has a smaller force resisting to bending (a smaller axial force) and is more likely to be bent according to an internal organ shape.

### [Operations of Stent Delivery System 150]

A stent detaining method using the endoscope system 300 including the stent delivery system 150 will be described below with reference to a procedure of detaining the stent device 100 in a biliary duct.

An operator inserts the insertion unit 210 of the endoscope 200 into a patient's body cavity from a natural opening such as a mouth. At this time, the operator operates the knob 223 or the like to bend the bending portion 212 according to necessity.

The operator inserts a guide wire into the treatment tool channel 230 of the endoscope 200 and inserts the guide wire into a biliary duct while observing the endoscope 200. Subsequently, the operator operates the guide wire to break through a stenosis region in the biliary duct under X-ray illumination and to move a distal end of the guide wire to a liver side from the stenosis region (a target position).

The operator inserts a proximal end of the guide wire protruding from the forceps plug 225 of the endoscope 200 into a through-hole of the tip 180 of the stent delivery system 150.

The operator pushes the stent delivery system 150 while holding the guide wire to cause the stent delivery system 150 to advance along the guide wire. The distal end of the stent delivery system 150 protrudes from the distal end of the treatment tool channel 230 of the endoscope 200. When the distal end of the stent delivery system 150 breaks through the stenosis region (a target position), the operator causes the stent delivery system 150 to advance or retract and determines a detainment position of the stent device 100. The operator may insert the stent delivery system 150 into the treatment tool channel 230 without using the guide wire.

When the target position of the stent deice 100 is determined, the operator causes the outer tubular member 160 to retract with respect to the inner tubular member 170. As a result, as illustrated in FIG. 1, the stent device 100 is gradually exposed from the distal side and expands.

When the stent device 100 is fully exposed, the stent device 100 expands as a whole, and the inner radius of the stent device 100 is greater than the outer radius of the inner tubular member 170. Therewith, the stent device 100 and the inner tubular member 170 are disengaged.

When the operator cause the inner tubular member 170 to retract after the stent device 100 and the inner tubular member 170 have been disengaged, the stent device 100 stays at the detainment position, and the inner tubular member 170 is removed from the stent device 100.

When the operator pulls out the stent delivery system 150 except the stent device 100 from the body, the procedure of detaining the stent device 100 ends.

The stent device 100 according to the present embodiment is likely to be bent even when the stent 110 is covered with the cover 120A or the like.

While the first embodiment has been described above in detail with reference to the drawings, any specific configuration is not limited to this embodiment and includes design changes or the like without departing from the gist of the present disclosure. Constituents described in the aforementioned embodiment and modified examples described below can be appropriately combined.

### Second Embodiment

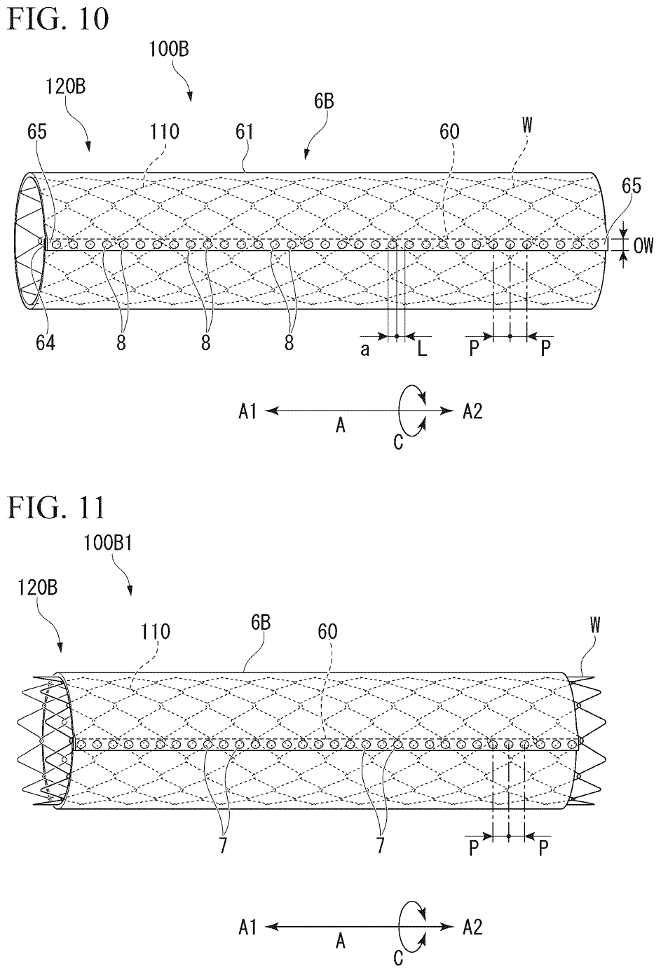

A second embodiment will be described below with reference to FIGS. 10 to 24. In the following description, the same constituents as described above will be referred to by the same reference signs, and repeated description thereof will be omitted.

### [Stent Device 100B]

FIG. 10 is a diagram illustrating a stent device 100B.

The stent device 100B is detained in a lumen of a digestive system such as a biliary duct, a gullet, a duodenum, a small intestine, or a large intestine and is mainly used to extend and maintain the lumen. The stent device 100B includes a stent 110 and a cover 120B. The stent device 100B is a covered stent in which the stent 110 formed by weaving wires is covered with the cover 120B.

The cover 120B includes an outer cover 6B disposed on the outside R2 in the radial direction R of the stent 110. The cover 120B does not include an inner cover.

The outer cover 6B is formed of a resin material having flexibility and is formed of, for example, an expanded polytetrafluoroethylene (ePTFE).

The outer cover 6B is disposed on the entire surface of the stent 110. Specifically, the outer cover 6B includes a body portion 61 disposed in both end portions and the central portion of the stent 110 and an overlapping bonding portion 60 extending in the longitudinal direction A.

The overlapping bonding portion 60 is a part in which one end portion 64 (hereinafter also referred to as a “first end portion 64”) and the other end portion 65 (hereinafter also referred to as a “second end portion 65”) in the circumferential direction C of the outer cover 6B overlap each other. An overlap width OW which is a length in the circumferential direction C of the overlapping bonding portion 60 is equal to or less than two times a cell width which is a length of a cell in the circumferential direction C and preferably ranges from 2 mm to 5 mm.

The outer cover 6B is not bonded on the entire surface of the stent 110 but is partially bonded. The first end portion 64 and the second end portion 65 are bonded at points. Points at which the end portions of the outer cover 6B are bonded at points are referred to as “bonding points 8.” The bonding points 8 are arranged with a pitch Pin the longitudinal direction A in the overlapping bonding portion 60. When the pitch P is equal to or greater than 5 mm, a gap is likely to be formed in the overlapping bonding portion 60 at the time of releasing or re-capturing the stent device 100B. Accordingly, the pitch P preferably ranges from 2 mm to 4 mm.

A length L of a non-bonded portion between neighboring bonding points 8 in the longitudinal direction A preferably satisfies 0<L≤3a. Here, a length a is the length of the bonding pint 8 in the longitudinal direction A. Since the bonding points 8 are cured to decrease flexibility and bendability of the stent device 100B, it is preferable that the neighboring bonding points 8 not be connected (0<L). Even when the stent device 100B is released or re-captured, it is preferable that the length L be equal to or less than three times the length a such that a gap is not formed in the overlapping bonding portion 60 (0<L≤3a).

Each bonding point 8 is formed in a shape such as a circle, an ellipse, or a quadrangle. Since the bonding points 8 are cured to decrease flexibility and bendability of the stent device 100B, the size of the bonding point 8 is preferably small and is, for example, more preferably smaller than the size of a cell. Even when the size of the bonding point 8 is greater than the size of a cell, it is preferable that the size of the bonding point 8 be smaller than the size of two cells.

FIG. 11 is a diagram illustrating a stent device 100B1 which is a modified example of the stent device 100B. The length in the longitudinal direction A of the outer cover 6B may be smaller than that of the stent 110. Both end portions of the stent 110 are exposed from the outer cover 6B.

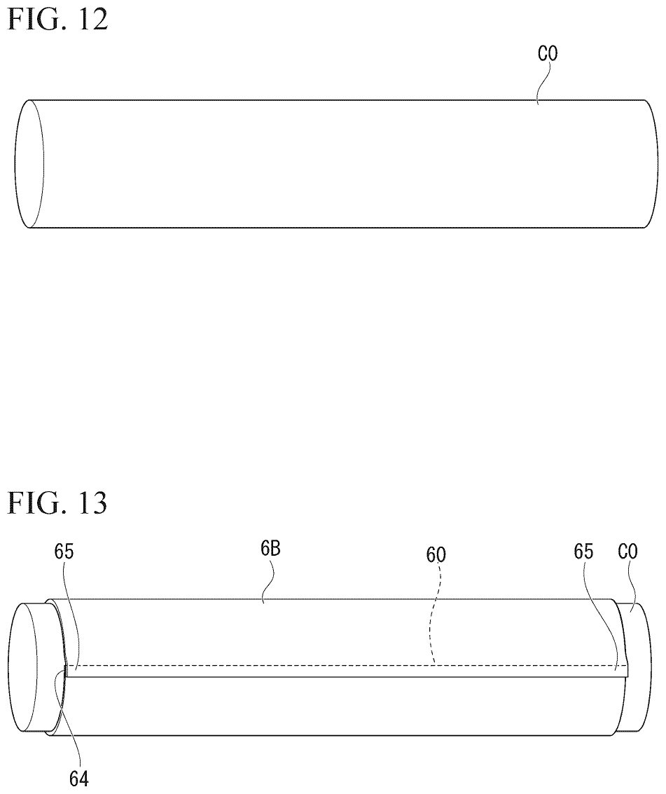

FIGS. 12 to 14 are diagrams illustrating a method of manufacturing the stent device 100B.

First, as illustrated in FIGS. 12 and 13, a sheet which will be the outer cover 6B is wound on a core rod CO. The first end portion 64 and the second end portion 65 are overlapped to form the overlapping bonding portion 60.

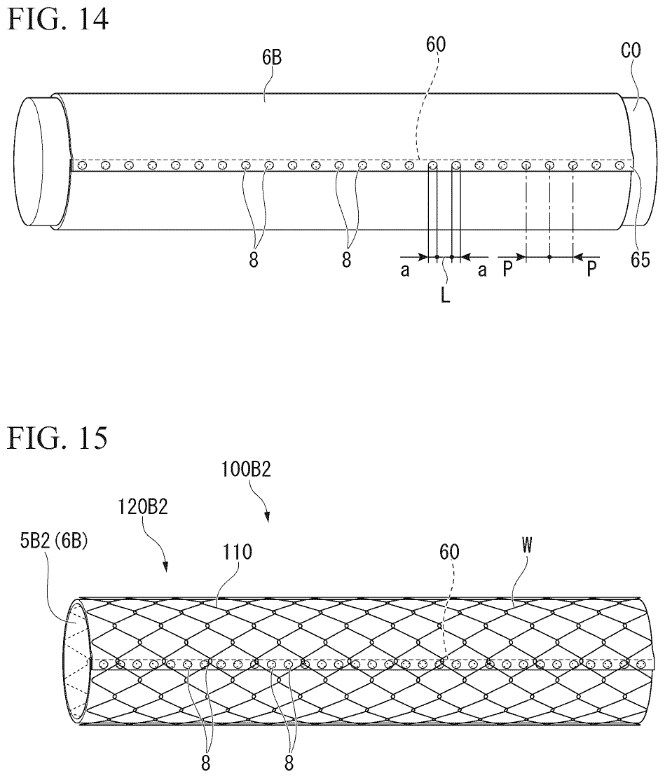

Then, as illustrated in FIG. 14, the overlapping bonding portion 60 in which the first end portion 64 and the second end portion 65 overlap is bonded at points. The point bonding is performed through thermocompression bonding or bonding using a joining material (such as PFA), or the like. The outer cover 6B is shaped into a tube.

Then, the outer cover 6B shaped into a tube is separated from the core rod CO, and the stent 110 is overlaid therewith. By fixing the outer cover 6B and the stent 110, the stent device 100B is completed. For example, by folding both end portions of the outer cover 6B to the inside of the stent 110 and bonding the end portions, the outer cover 6B and the stent 110 are fixed. Alternatively, the outer cover 6B and the stent 110 may be fixed in an intermediate portion in the longitudinal direction A. In this case, a fixing method not hindering bendability of the stent 110 can be preferably used.

Even when the stent device 100B is released or re-captured, the stent device 100B including the cover 120B can prevent a gap from being formed in the outer cover 6B, appropriately prevent infiltration of biological tissue, and maintain flexibility and bendability.

### <Stent Device 100B2>

FIG. 15 is a diagram illustrating a stent device 100B2 which is a modified example of the stent device 100B. The stent device 100B2 includes a stent 110 and a cover 120B2.

The cover 120B2 includes an inner cover 5B2 disposed on the inside R1 in the radial direction R of the stent 110. The cover 120B2 does not include an outer cover. The inner cover 5B2 is the same as the outer cover 6B. By covering the stent 110 with the outer cover 6B which is formed as illustrated in FIG. 14, the stent device 100B2 can be formed.

### <Stent Device 100B3>

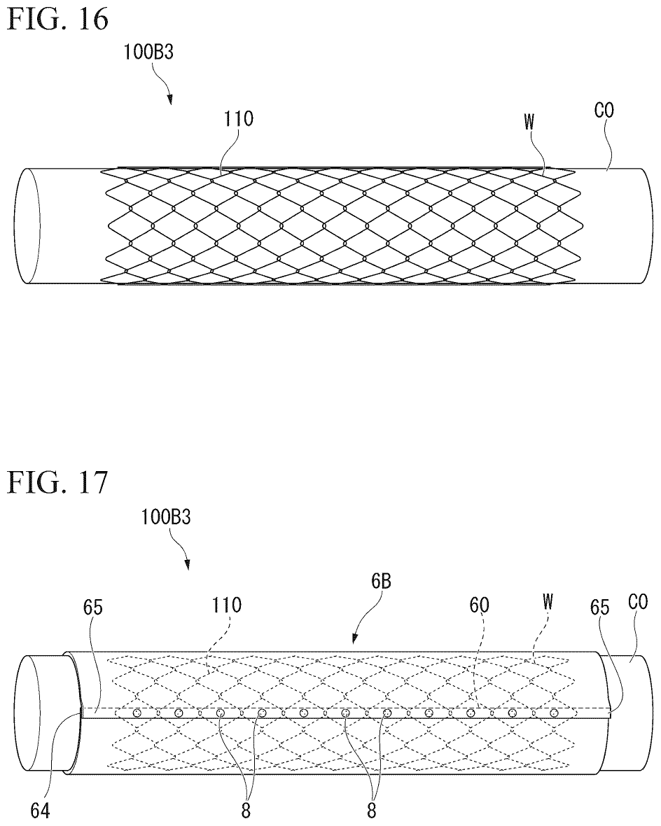

FIGS. 16 to 18 are diagrams illustrating a method of manufacturing a stent device 100B3 which is a modified example of the stent device 100B. First, as illustrated in FIG. 16, the stent 110 is wound on a core rod CO. A cell pitch of the stent 110 is equal to or less than 4 mm and is, for example, 3 mm.

Then, as illustrated in FIG. 17, a sheet which will be the outer cover 6B is wound on the stent 110 and is shaped into a tube. In view of easiness of bonding, it is preferable that the bonding points 8 be formed in areas in which no wire W is located (areas in which no cell is located) when seen in the radial direction R. For example, when the cell pitch of the stent 110 is 3 mm, the pitch P between the bonding points 8 is substantially 3 mm.

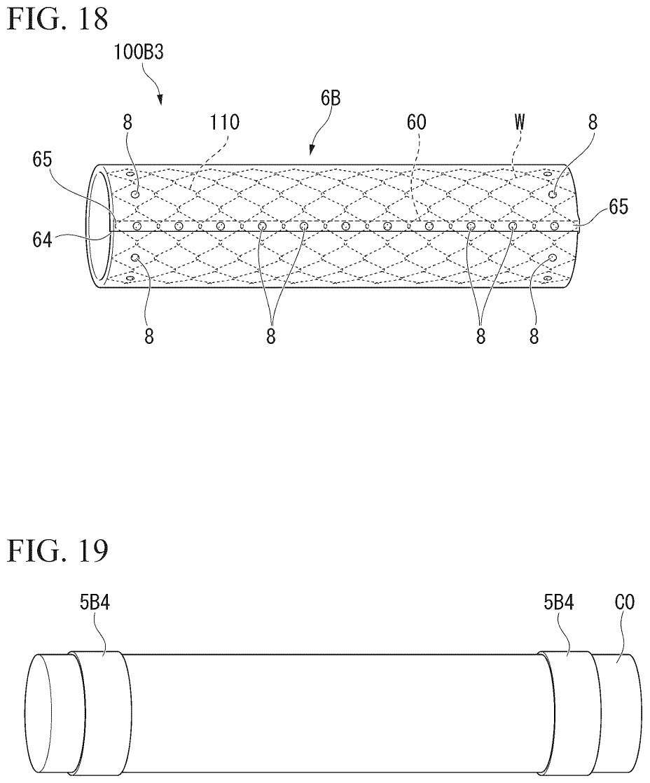

Then, as illustrated in FIG. 18, the stent 110 and the outer cover 6B shaped into a tube are separated from the core rod CO, and the outer cover 6B and the stent 110 are fixed, whereby the stent device 100B3 is completed. By folding both end portions of the outer cover 6B to the inside of the stent 110 and bonding the end portions at points, the outer cover 6B and the stent 110 are fixed.

### <Stent Device 100B4>

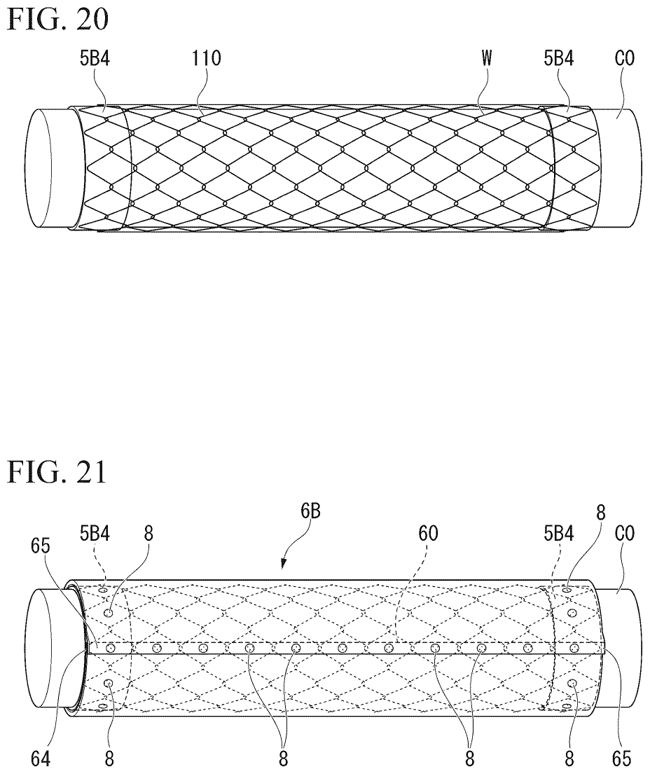

FIGS. 19 to 22 are diagrams illustrating a method of manufacturing a stent device 100B4 which is a modified example of the stent device 100B. First, as illustrated in FIG. 19, inner covers 5B4 are wound on both end portions of a core rod CO. Then, as illustrated in FIG. 20, the stent 110 is wound on the inner covers 5B4. The inner covers 5B4 are located in both end portions of the stent 110. A cell pitch of the stent 110 is equal to or less than 4 mm and is, for example, 3 mm.

Then, as illustrated in FIG. 21, a sheet which will be the outer cover 6B is wound on the stent 110 and is shaped into a tube. The inner covers 5B4, the outer cover 6B, and the stent 110 are fixed by bonding the inner covers 5B4 and the outer cover 6B at points with cells interposed therebetween.

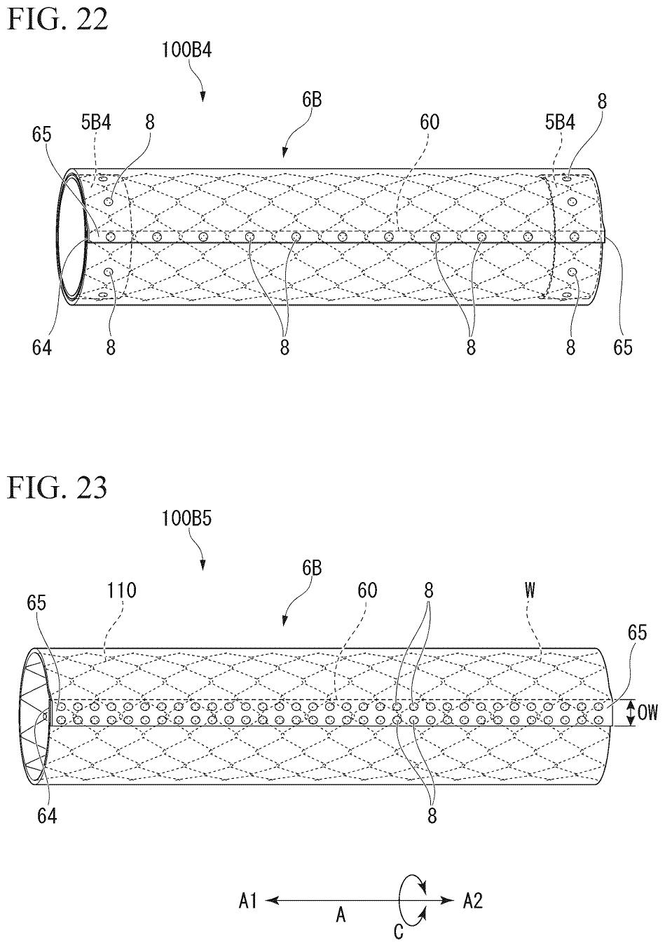

Then, as illustrated in FIG. 22, the stent 110, the inner covers 5B4, and the outer cover 6B shaped into a tube are separated from the core rod CO, whereby the stent device 100B4 is completed.

### <Stent Device 100B5>

FIG. 23 is a diagram illustrating a stent device 100B5 which is a modified example of the stent device 100B. In the stent device 100B5, the bonding points 8 are arranged in two lines (arranged in parallel) in the longitudinal direction A in the overlapping bonding portion 60. An overlap width OW is preferably one time to two times the length in the circumferential direction C of a cell.

The stent device 100B5 has a larger number of bonding points 8 than that of the stent device 100B. Accordingly, even when the stent device is released or re-captured, it is possible to prevent a gap from being formed in the outer cover 6B and to appropriately prevent infiltration of biological tissue. With the stent device 100B5, it is possible to curb a local increase in thickness of the outer cover 6B due to reduction of the outer cover 6B to form wrinkles. As a result, it is possible to curb an increase in slide resistance and an increase in an operating force at the time of release.

### <Stent Device 100B6>

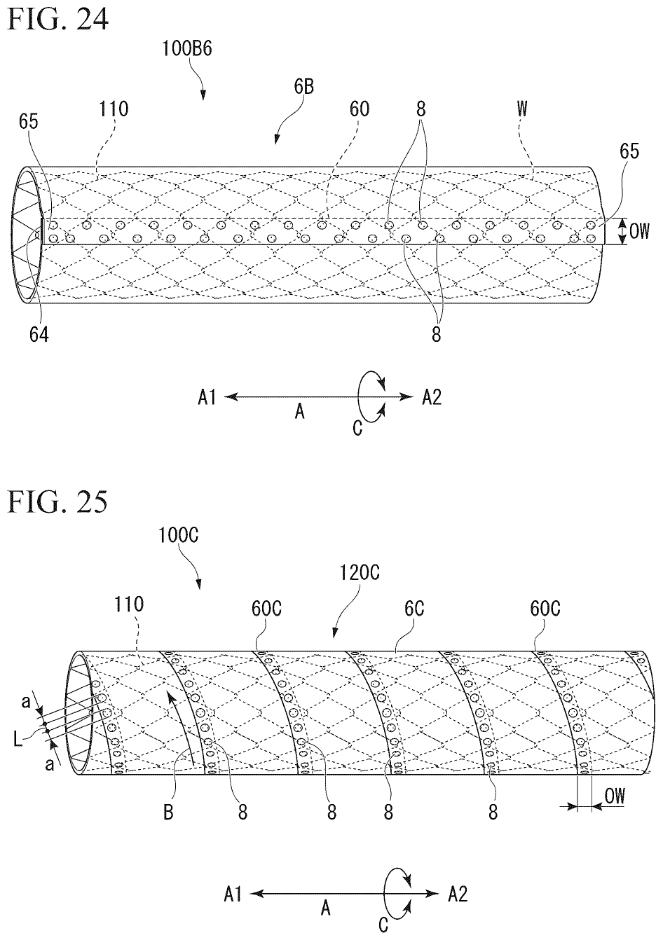

FIG. 24 is a diagram illustrating a stent device 100B6 which is a modified example of the stent device 100B. In the stent device 100B6, the bonding points 8 are alternately arranged in two lines (arranged in zigzag) in the longitudinal direction A in the overlapping bonding portion 60. The overlap width OW is preferably one time to two times the length in the circumferential direction C of a cell.

The stent device 100B6 has a larger number of bonding points 8 than that of the stent device 100B. Accordingly, even when the stent device is released or re-captured, it is possible to prevent a gap from being formed in the outer cover 6B and to appropriately prevent infiltration of biological tissue. With the stent device 100B6, it is possible to curb a local increase in thickness of the outer cover 6B due to reduction of the outer cover 6B to form wrinkles. As a result, it is possible to avoid an increase in slide resistance and an increase in an operating force at the time of release.

The stent device 100B according to the present embodiment is likely to be bent even when the stent 110 is covered with the cover 120B or the like.

While the second embodiment has been described above in detail with reference to the drawings, any specific configuration is not limited to this embodiment and includes design changes or the like without departing from the gist of the present disclosure. Constituents described in the aforementioned embodiment and modified examples described below can be appropriately combined.

### Third Embodiment

A third embodiment will be described below with reference to FIGS. 25 to 28. In the following description, the same constituents as described above will be referred to by the same reference signs, and repeated description thereof will be omitted.

### [Stent Device 100C]

FIG. 25 is a diagram illustrating a stent device 100C.

The stent device 100C is detained in a lumen of a digestive system such as a biliary duct, a gullet, a duodenum, a small intestine, or a large intestine and is mainly used to extend and maintain the lumen. The stent device 100C includes a stent 110 and a cover 120C. The stent device 100C is a covered stent in which the stent 110 formed by weaving wires is covered with the cover 120C.

The cover 120C includes an outer cover 6C disposed on the outside R2 in the radial direction R of the stent 110. The cover 120C does not include an inner cover.

The outer cover 6C is wound in a spiral shape in the longitudinal direction A. The outer cover 6C includes an overlapping bonding portion 60C.

The overlapping bonding portion 60C is a portion in which the outer cover 6C overlaps and is formed in a spiral shape. The overlap width OW of the overlapping bonding portion 60C ranges from 2 mm to 10 mm and preferably ranges from 3 mm to 6 mm.

The outer cover 6C is not bonded on the entire surface of the stent 110 and is partially bonded. The overlapping end portions of the outer cover 6C are bonded at points in the overlapping bonding portion 60C. The bonding points 8 are arranged with a predetermined pitch in a spiral direction B in which the overlapping bonding portion 60C extends.

A length L of a non-bonded portion between neighboring bonding points 8 in the spiral direction B preferably satisfies 0<L≤3a. Here, the length a is a length of a bonding point 8 in the spiral direction B.

In the overlapping bonding portion 60C, the bonding points 8 may be arranged in two lines (arranged in parallel, arranged in zigzag) in the spiral direction B. A gap is less likely to be formed in the overlapping bonding portion 60C.

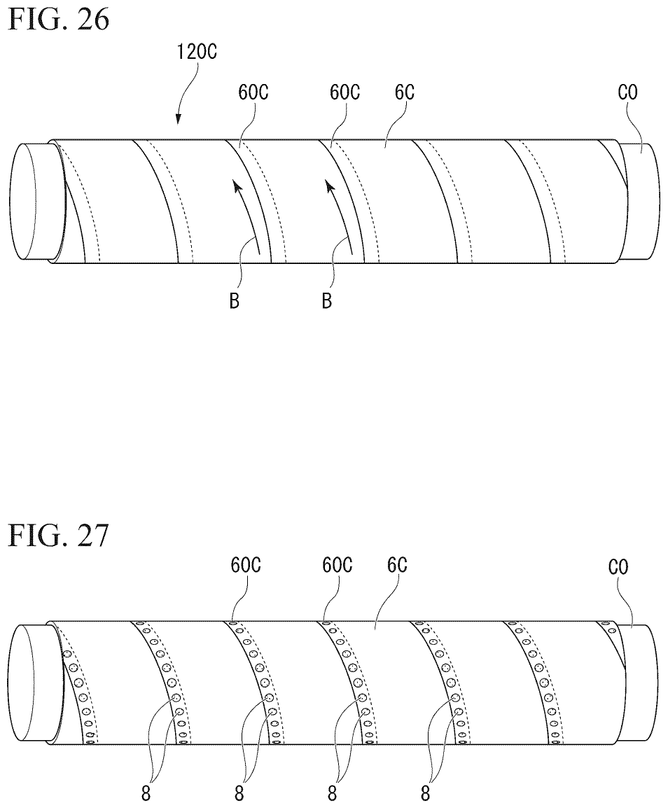

FIGS. 26 and 27 are diagrams illustrating a method of manufacturing the stent device 100C.

First, as illustrated in FIG. 26, a sheet which will be the outer cover 6C is wound on a core rod CO. The end portions of the outer cover 6C are overlapped to form the overlapping bonding portion 60C.

Then, as illustrated in FIG. 27, the overlapping bonding portion 60C is bonded at points. The point bonding is performed through thermocompression bonding or bonding using a joining material (such as PFA), or the like. The outer cover 6C is shaped into a tube.

Then, the outer cover 6C shaped into a tube is separated from the core rod CO, and the stent 110 is overlaid therewith. By fixing the outer cover 6C and the stent 110, the stent device 100C is completed. At this time, the bonding points 8 of the overlapping bonding portion 60C are adjusted to be located at positions separated from the hooked portions 2 of the stent 110.

In the stent device 100C including the cover 120C, the outer cover 6C is shaped into a tube by winding the outer cover 6C in a spiral shape and bonding the overlapping bonding portion 60C at points. Accordingly, with the stent device 100C, when the stent 110 is woven in a spiral shape, it is possible to arrange the bonding points 8 to correspond to meshes of the wires W and to reduce an overlapping portion between the hooked portions 2 and the boding points 8. As a result, it is possible to curb an increase in slide resistance and an increase in an operating force at the time of release.

### <Stent Device 100C1>

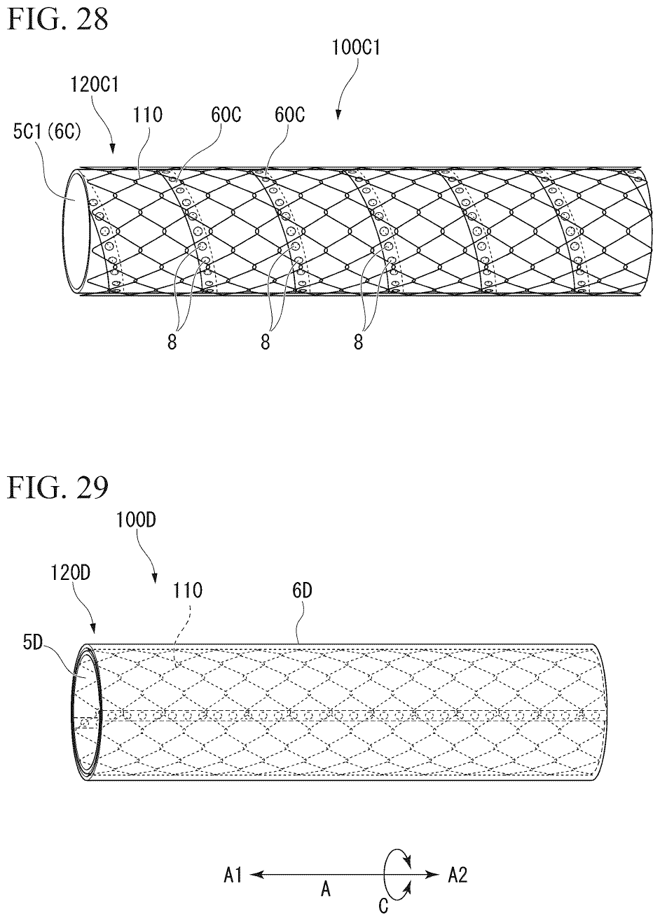

FIG. 28 is a diagram illustrating a stent device 100C1 which is a modified example of the stent device 100C. The stent device 100C1 includes a stent 110 and a cover 120C1.

The cover 120C1 includes an inner cover 5C1 disposed on the inside R1 in the radial direction R of the stent 110. The cover 120C1 does not include an outer cover. The inner cover 5C1 is the same as the outer cover 6C. The stent device 100C1 can be formed by overlaying the inner cover 5C1 formed as illustrated in FIG. 27 with the stent 110.

The stent device 100C according to the present embodiment is likely to be bent even when the stent 110 is covered with the cover 120C or the like.

While the third embodiment has been described above in detail with reference to the drawings, any specific configuration is not limited to this embodiment and includes design changes or the like without departing from the gist of the present disclosure. Constituents described in the aforementioned embodiment and modified examples described below can be appropriately combined.

### Fourth Embodiment

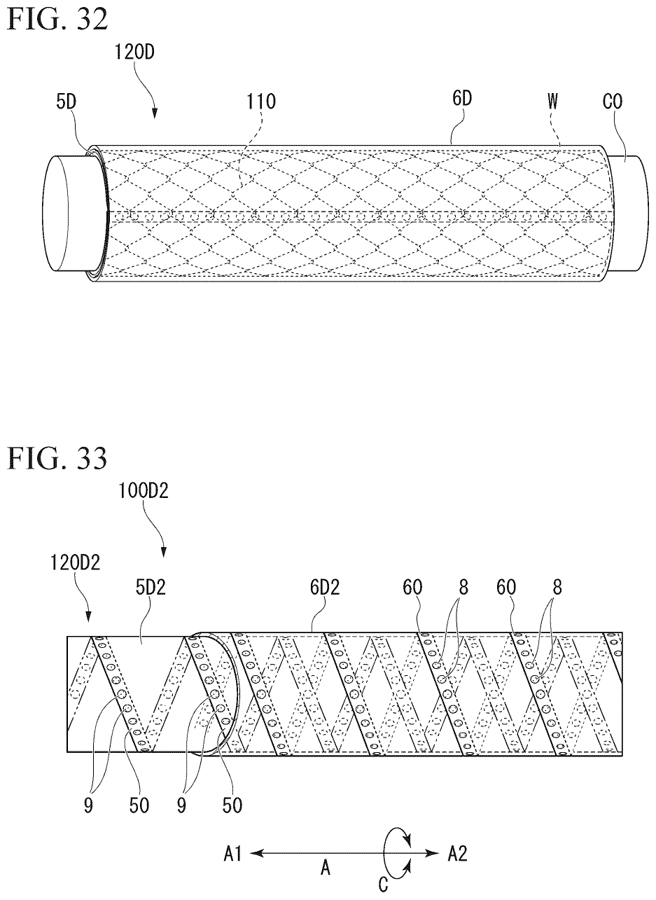

A fourth embodiment will be described below with reference to FIGS. 29 to 35. In the following description, the same constituents as described above will be referred to by the same reference signs, and repeated description thereof will be omitted.

### [Stent Device 100D]

FIG. 29 is a diagram illustrating a stent device 100D.

The stent device 100D is detained in a lumen of a digestive system such as a biliary duct, a gullet, a duodenum, a small intestine, or a large intestine and is mainly used to extend and maintain the lumen. The stent device 100D includes a stent 110 and a cover 120D. The stent device 100D is a covered stent in which the stent 110 formed by weaving wires is covered with the cover 120D.

The cover 120D is formed by overlapping an inner cover 5D disposed on the inside R1 in the radial direction R of the stent 110 and an outer cover 6D disposed on the outside R2 in the radial direction R of the stent 110.

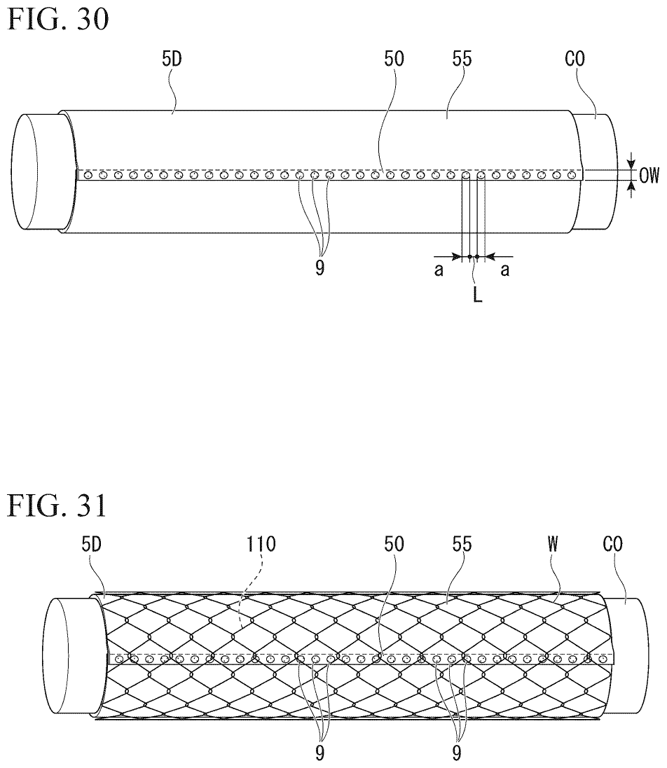

The inner cover 5D is disposed on the entire surface of the stent 110. Specifically, the inner cover 5D includes a body portion 55 disposed in both end portions and the central portion of the stent 110 and an overlapping bonding portion 50 extending in the longitudinal direction A (see FIG. 30).

The overlapping bonding portion 50 is a portion in which the inner cover 5D overlaps similarly to the overlapping bonding portion 60 according to the second embodiment. An overlap width OW which is a length in the circumferential direction C of the overlapping bonding portion 50 ranges from 1 mm to 10 mm and preferably ranges from 3 mm to 6 mm.

The inner cover 5D and the outer cover 6D are not bonded on the entire surface of the stent 110 but are partially bonded. The overlapping end portions 5D of the inner cover 5D are bonded at points in the overlapping bonding portion 50. Points at which the end portions of the inner cover 5D are bonded at points are referred to as “bonding points 9.”

A length L of a non-bonded portion between neighboring bonding points 9 in the longitudinal direction A preferably satisfies 0<L≤3a. Here, a length a is the length of the bonding point 9 in the longitudinal direction A and is, for example, equal to or greater than 0.5 mm.

FIGS. 30 to 32 are diagrams illustrating a method of manufacturing the stent device 100D.

First, as illustrated in FIG. 30, a sheet which will be the inner cover 5D is wound on a core rod CO. The end portions of the inner cover 5D are overlapped to form the overlapping bonding portion 50.

Then, the overlapping bonding portion 50 is bonded at points. The point bonding is performed through thermocompression bonding or bonding using a joining material (such as PFA), or the like. The inner cover 5D is shaped into a tube.

Then, as illustrated in FIG. 31, the stent 110 is wound on the inner cover 5D shaped into a tube. Then, the outer cover 6D shaped into a tube in the same way as the outer cover 6B is wound on the stent 110.

Then, the inner cover 5D and the outer cover 6D are bonded, and the inner cover 5D, the outer cover 6D, and the stent 110 are fixed. Then, the inner cover 5D, the outer cover 6D, and the stent 110 are separated from the core rod CO, and the stent device 100D is completed.

It is preferable that the cover 120D be disposed such that the overlapping bonding portion 50 of the inner cover 5D and the overlapping bonding portion 60 of the outer cover 6D do not overlap. It is preferable that the cover 120D be disposed such that an area in which the overlapping bonding portion 50, the overlapping bonding portion 60, and the hooked portions 2 of the stent 110 overlap decreases. This is because an area in which they overlap to increase partially in thickness can be reduced. When the stent device 100D is accommodated in the stent delivery system 150, it is possible to curb an increase in an accommodation outer diameter of the stent device 100D and an increase in operating force.

### <Stent Device 100D2>

FIG. 33 is a diagram illustrating a stent device 100D2 which is a modified example of the stent device 100D. The stent device 100D2 includes a stent 110 and a cover 120D2. The stent 110 is not illustrated in FIG. 33.

The cover 120D2 is formed by overlapping an inner cover 5D2 disposed on the inside R1 in the radial direction R of the stent 110 and an outer cover 6D2 disposed on the outside R2 in the radial direction R of the stent 110.

The inner cover 5D2 and the outer cover 6D2 are covers which are wound in a spiral shape and shaped into a tube, similarly to the outer cover 6C according to the third embodiment. The spiral direction of the inner cover 5D2 wound in a spiral shape and the spiral direction of the outer cover 6C2 wound in a spiral shape are the same direction.

It is preferable that the cover 120D2 be disposed such that an area in which the overlapping bonding portion 50 of the inner cover 5D2 and the overlapping bonding portion 60 of the outer cover 6D2 overlap decreases.

### <Stent Device 100D3>

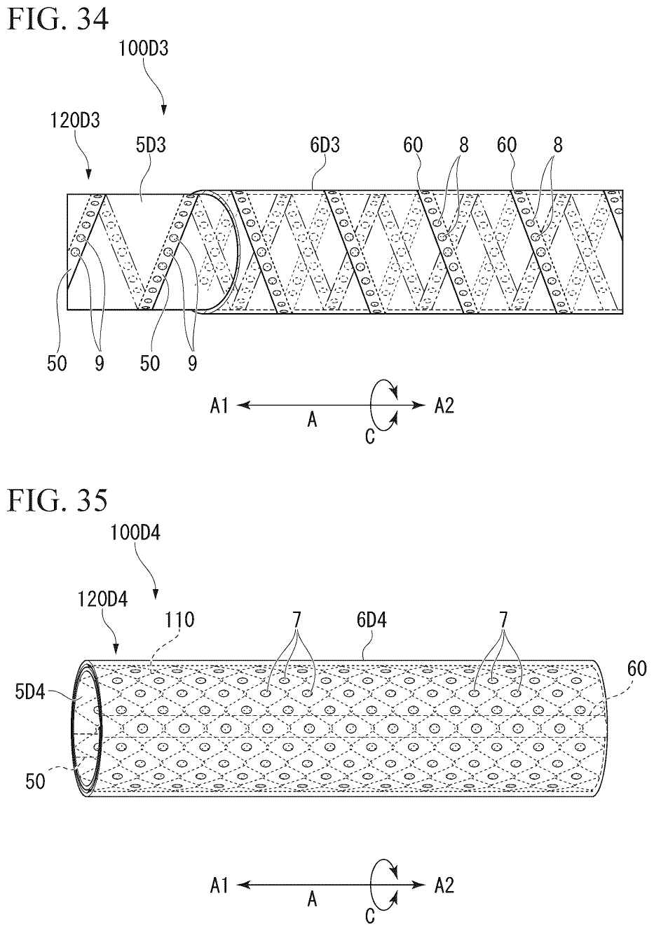

FIG. 34 is a diagram illustrating a stent device 100D3 which is a modified example of the stent device 100D. The stent device 100D3 includes a stent 110 and a cover 120D3. The stent 110 is not illustrated in FIG. 34.

The cover 120D3 is formed by overlapping an inner cover 5D3 disposed on the inside R1 in the radial direction R of the stent 110 and an outer cover 6D3 disposed on the outside R2 in the radial direction R of the stent 110.

The inner cover 5D3 and the outer cover 6D3 are covers which are wound in a spiral shape and shaped into a tube, similarly to the outer cover 6C according to the third embodiment. The spiral direction of the inner cover 5D3 wound in a spiral shape and the spiral direction of the outer cover 6D3 wound in a spiral shape are opposite to each other.

### <Stent Device 100D4>

FIG. 35 is a diagram illustrating a stent device 100D4 which is a modified example of the stent device 100D. The stent device 100D4 includes a stent 110 and a cover 120D4.

The cover 120D4 is formed by overlapping an inner cover 5D4 disposed on the inside R1 in the radial direction R of the stent 110 and an outer cover 6D4 disposed on the outside R2 in the radial direction R of the stent 110.

The inner cover 5D4 and the outer cover 6D4 are covers which are shaped into a tube, similarly to the outer cover 6B according to the second embodiment. The inner cover 5D4 and the outer cover 6D4 are bonded at points with spaces (cells) surrounded with wires W interposed therebetween. The inner cover 5D4 and the outer cover 6D4 are bonded at points with most cells of the stent 110 interposed therebetween.

The stent device 100D according to the present embodiment is likely to be bent even when the stent 110 is covered with the cover 120D or the like.

While the fourth embodiment has been described above in detail with reference to the drawings, any specific configuration is not limited to this embodiment and includes design changes or the like without departing from the gist of the present disclosure. Constituents described in the aforementioned embodiment and modified examples described below can be appropriately combined.

### Fifth Embodiment

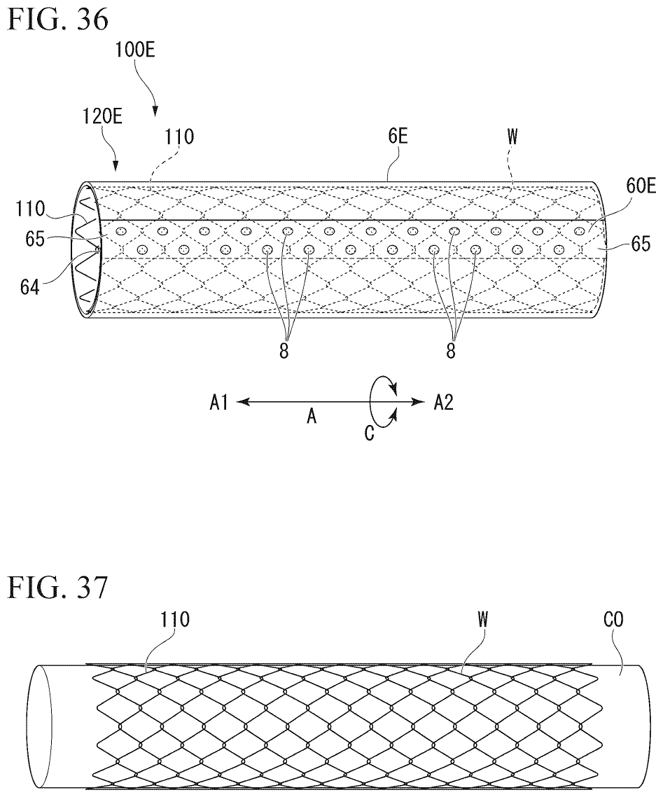

A fifth embodiment will be described below with reference to FIGS. 36 to 40. In the following description, the same constituents as described above will be referred to by the same reference signs, and repeated description thereof will be omitted.

### [Stent Device 100E]

FIG. 36 is a diagram illustrating a stent device 100E.

The stent device 100E is detained in a lumen of a digestive system such as a biliary duct, a gullet, a duodenum, a small intestine, or a large intestine and is mainly used to extend and maintain the lumen. The stent device 100E includes a stent 110 and a cover 120E. The stent device 100E is a covered stent in which the stent 110 formed by weaving wires is covered with the cover 120E.

The cover 120E includes an outer cover 6E disposed on the outside R2 in the radial direction R of the stent 110. The cover 120E does not include an inner cover.

The outer cover 6E is formed of a resin material having flexibility and is formed of, for example, an expanded polytetrafluoroethylene (ePTFE).

The outer cover 6E is disposed on the entire surface of the stent 110. Specifically, the outer cover 6E includes a body portion 61 disposed in both end portions and the central portion of the stent 110 and an overlapping bonding portion 60E extending in the longitudinal direction A.

The overlapping bonding portion 60E is a portion in which one end portion 64 (hereinafter also referred to as a “first end portion 64”) and the other end portion 65 (hereinafter also referred to as a “second end portion 65”) in the circumferential direction C of the outer cover 6E overlap each other.

The outer cover 6E is not bonded on the entire surface of the stent 110 but is partially bonded. The first end portion 64 and the second end portion 65 are bonded at points. The bonding points 8 are alternately arranged in two lines (arranged in zigzag) in the longitudinal direction A in the overlapping bonding portion 60. The bonding points 8 in one line and the bonding points 8 in the other line are alternately arranged in the longitudinal direction A. The bonding points 8 are formed in areas in which no wire W is located (areas in which cells are located) when seen in the radial direction R.

FIGS. 37 to 41 are diagrams illustrating a method of manufacturing the stent device 100E.

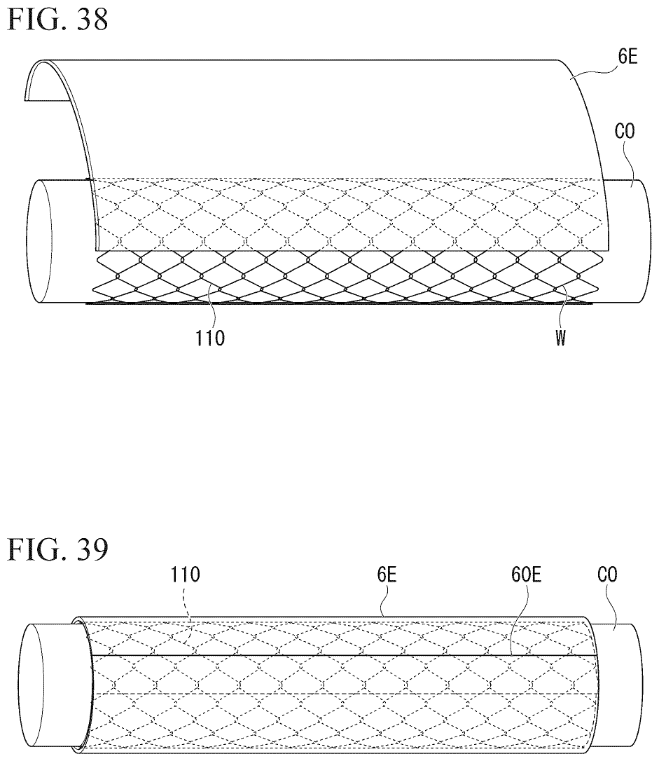

First, as illustrated in FIG. 37, the stent 110 is wound on a core rod CO. Then, as illustrated in FIG. 38, a sheet which will be the outer cover 6E is wound on the stent 110. Then, as illustrated in FIG. 39, the first end portion 64 and the second end portion 65 are overlapped to form the overlapping bonding portion 60E.

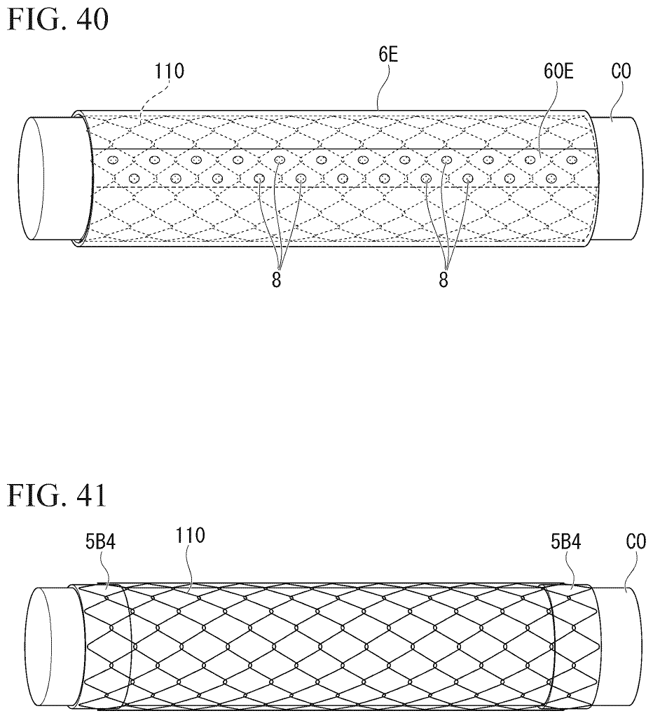

Then, as illustrated in FIG. 40, the first end portion 64 and the second end portion 65 are bonded at points, and the outer cover 6E and the stent 110 are fixed. Then, the stent 110 and the outer cover 6E shaped into a tube are separated from the core rod CO, and the stent device 100E is completed. Both end portions of the outer cover 6E may be folded to the inside of the stent 110 and bonded at points.

As illustrated in FIG. 41, the inner cover 5B4 of the cover 120E may be disposed on the inside of the stent 110, and the inner cover 5B4 and the outer cover 6E may be bonded at points.

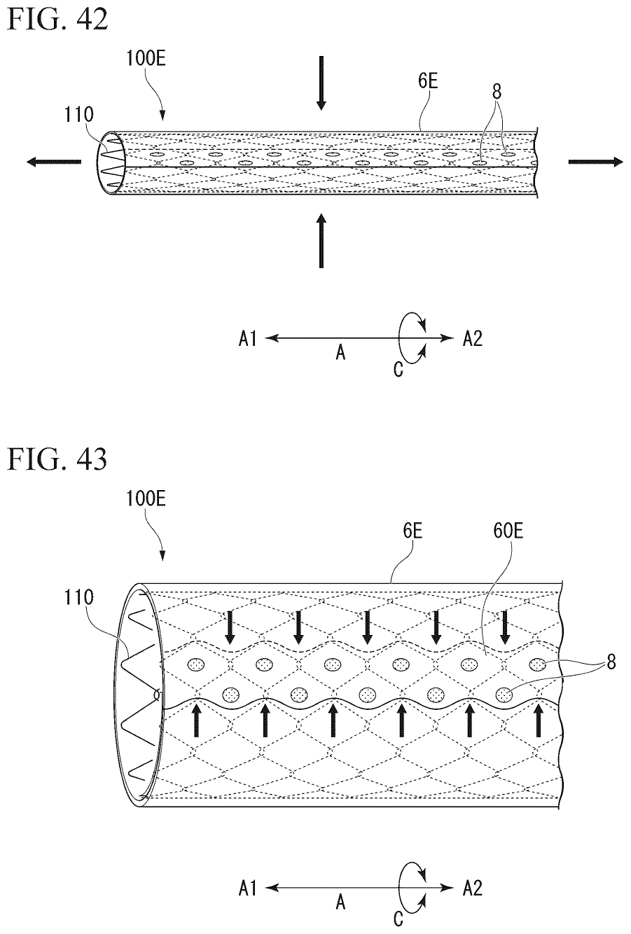

FIGS. 42 and 43 are diagrams illustrating operations and effects of the stent device 100E. By arranging the bonding points 8 in zigzag in two lines, since a radius decreases and a stent length increases when the stent device 100E decrease in radius and are accommodated in the stent delivery system 150 as illustrated in FIG. 42, a cover length of the outer cover 6E increases and a length in the circumferential direction C of the outer cover 6E decreases. On the other hand, when the stent is released from the stent delivery system 150, the bonding points 8 move laterally with expansion of the stent device 100E, and thus a non-bonded portion of the overlapping bonding portion 60E is attracted in an arrow direction. A length decreasing in the circumferential direction C when the stent device 100E decrease in radius is complemented when the stent device 100E expands. As a result, it is possible to prevent the stent device 100E from decreasing in radius by the cover 120E.

Since the bonding points 8 are arranged in zigzag in two lines, an extending force applied to the bonding points 8 at the time of decreasing in radius is likely to be distributed to the bonding points 8 in the other line. As a result, it is possible to prevent breakage which is likely to occur at a bonding boundary of the overlapping bonding portion 60E.

### <Stent Device 100E1>

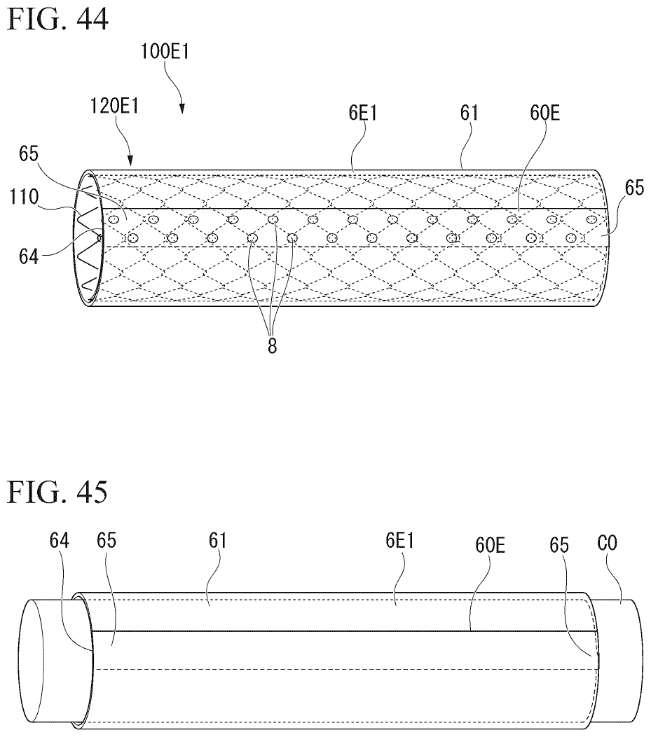

FIG. 44 is a diagram illustrating a stent device 100E1 which is a modified example of the stent device 100E. The stent device 100E1 is different from the stent device 100E in that the bonding points 8 are not necessarily disposed in areas in which no wire W is located (areas in which cells are located). The stent device 100E1 includes a stent 110 and a cover 120E1.

The cover 120E1 includes an outer cover 6E1 disposed on the outside R2 in the radial direction R of the stent 110. The cover 120E1 does not include an inner cover.

The outer cover 6E1 is disposed on the entire surface of the stent 110. Specifically, the outer cover 6E1 includes a body portion 61 disposed in both end portions and the central portion of the stent 110 and an overlapping bonding portion 60E extending in the longitudinal direction A.

In the overlapping bonding portion 60E, the bonding points 8 are alternately arranged in two lines (arranged in zigzag) in the longitudinal direction A. The bonding points 8 are not necessary disposed in areas in which no wire W is located (areas in which cells are located).

FIGS. 45 to 47 are diagrams illustrating a method of manufacturing the stent device 100E1.

First, as illustrated in FIG. 45, a sheet which will be the outer cover 6E1 is wound on a core rod CO. The first end portion 64 and the second end portion 65 are overlapped to form the overlapping bonding portion 60E.

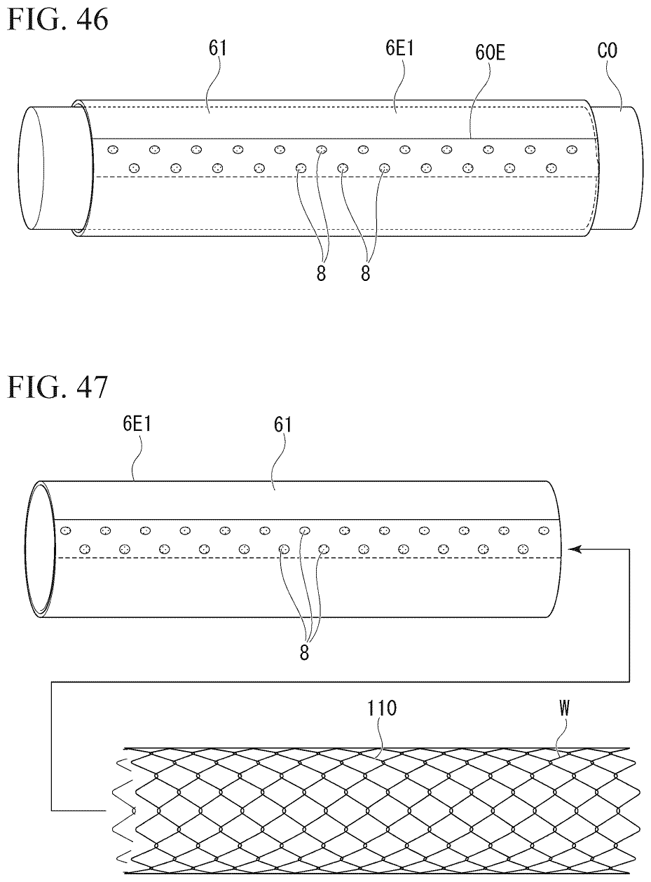

Then, as illustrated in FIG. 46, the overlapping bonding portion 60E in which the first end portion 64 and the second end portion 65 overlap is bonded at points. The point bonding is performed through thermocompression bonding or bonding using a joining material (such as PFA), or the like. The outer cover 6E1 is shaped into a tube.

Then, the outer cover 6E1 shaped into a tube is separated from the core rod CO, and the stent 110 is overlaid therewith. By fixing the outer cover 6E1 and the stent 110, the stent device 100E1 is completed.

In the stent device 100E1, the bonding points 8 can be disposed at arbitrary positions according to usage. In the stent device 100E1, the bonding points 8 may not be necessarily disposed in areas in which no wire W is located (areas in which cells are located).

The stent device 100E according to the present embodiment is likely to be bent even when the stent 110 is covered with the cover 120E or the like.

While the fifth embodiment has been described above in detail with reference to the drawings, any specific configuration is not limited to this embodiment and includes design changes or the like without departing from the gist of the present disclosure. Constituents described in the aforementioned embodiment and modified examples described below can be appropriately combined.

### Sixth Embodiment

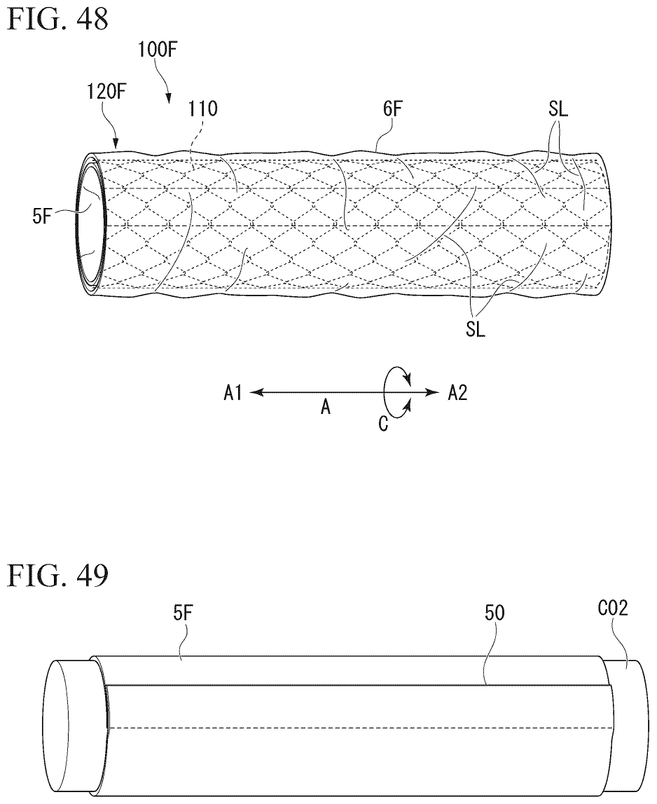

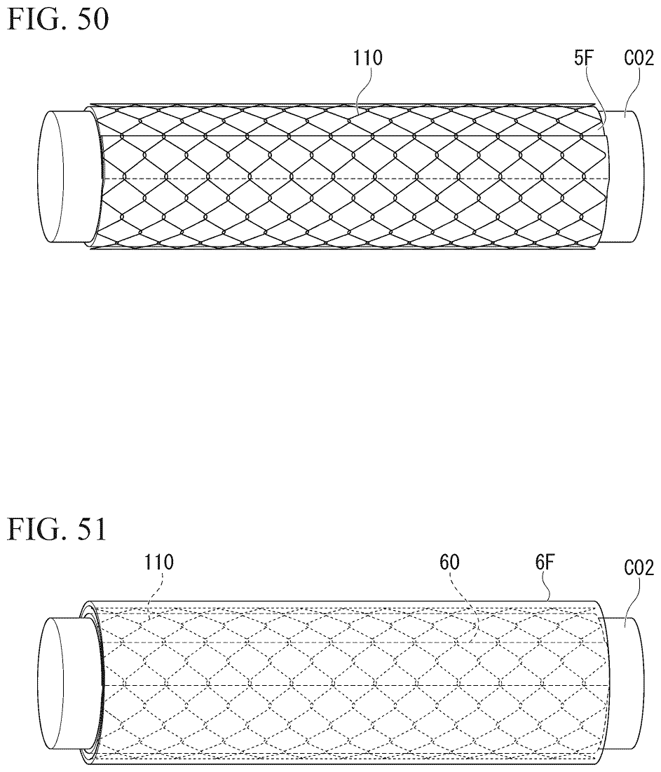

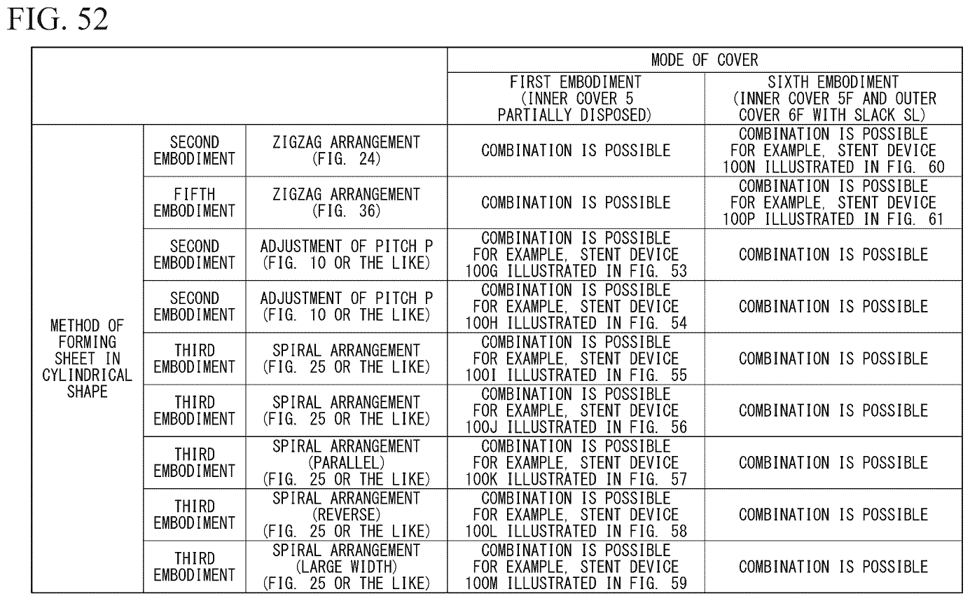

A sixth embodiment will be described below with reference to FIGS. 48 to 51.

In the following description, the same constituents as described above will be referred to by the same reference signs, and repeated description thereof will be omitted.

### [Stent Device 100F]

FIG. 48 is a diagram illustrating a stent device 100F.

The stent device 100F is detained in a lumen of a digestive system such as a biliary duct, a gullet, a duodenum, a small intestine, or a large intestine and is mainly used to extend and maintain the lumen. The stent device 100F includes a stent 110 and a cover 120F. The stent device 100F is a covered stent in which the stent 110 formed by weaving wires is covered with the cover 120F.

The cover 120F is formed by overlapping an inner cover 5F disposed on the inside R1 in the radial direction R of the stent 110 and an outer cover 6F disposed on the outside R2 in the radial direction R of the stent 110.

The inner cover 5F and the outer cover 6F have slack (looseness) SL with respect to the stent 110.

FIGS. 49 to 51 are diagrams illustrating a method of manufacturing the stent device 100F.

First, as illustrated in FIG. 49, a sheet which will be the inner cover 5F is wound on a core rod CO2. An outer radius of the core rod CO2 is slightly larger than an outer radius of the stent 110. Then, the overlapping bonding portion 50 is bonded, and thus the inner cover 5D is shaped into a tube.

Then, as illustrated in FIG. 50, the stent 110 is wound on the inner cover 5F shaped into a tube. Since the outer radius of the core rod CO2 is slightly larger than the outer radius of the stent 110, the stent 110 is wound on the core rod CO2 from above the inner cover 5F in a state in which the stent has increased slightly in radius.

Then, the outer cover 6F shaped into a tube in the same way as the outer cover 6B is wound on the stent 110.

Then, the inner cover 5F and the outer cover 6F are bonded, and the inner cover 5F, the outer cover 6F, and the stent 110 are fixed. Then, the inner cover 5F, the outer cover 6F, and the stent 110 are separated from the core rod CO2, and the stent device 100F is completed.

In the aforementioned manufacturing method, the outer radius of the core rod CO2 is slightly larger than the outer radius of the stent 110. When the stent device 100F is separated from the core rod CO2, the stent 110 decrease in radius, and thus slack SL with respect to the stent 110 is caused in the inner cover 5F and the outer cover 6F.

Even when the stent 110 is covered with the cover 120F, slack SL is caused in the inner cover 5F and the outer cover 6F, and thus the stent device 100F according to the present embodiment is likely to be bent.

While the sixth embodiment has been described above in detail with reference to the drawings, any specific configuration is not limited to this embodiment and includes design changes or the like without departing from the gist of the present disclosure. Constituents described in the aforementioned embodiment and modified examples described below can be appropriately combined.

### Seventh Embodiment

A seventh embodiment will be described below with reference to FIGS. 52 to 61. The seventh embodiment is an embodiment in which the first to sixth embodiments described above are combined. In the following description, the same constituents as described above will be referred to by the same reference signs, and repeated description thereof will be omitted.

FIG. 52 is a diagram illustrating an example of a combination of the embodiments.

The methods of shaping the covers into a cylindrical shape and the aspects of the covers which are described above in the first to sixth embodiments can be combined as illustrated in FIG. 52.

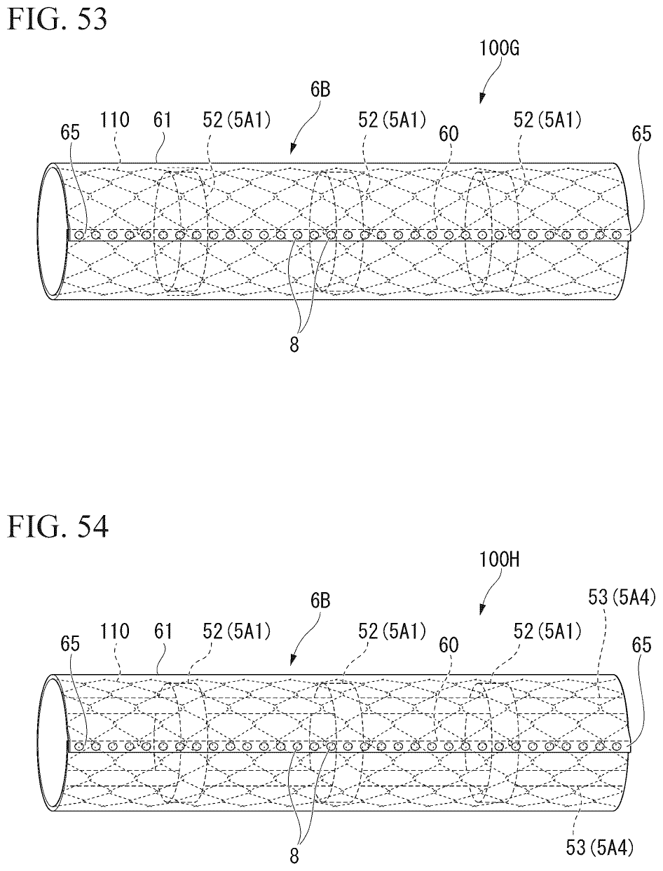

FIG. 53 is a diagram illustrating a stent device 100G which is an example of a combination.

The stent device 100G includes a stent 110, an inner cover 5A1, and an outer cover 6B. Since bonding at the time of shaping the inner cover 5A1 into a cylindrical shape can be omitted, it is possible to easily manufacture the stent device 100G.

FIG. 54 is a diagram illustrating a stent device 100H which is an example of a combination.

The stent device 100H includes a stent 110, an inner cover 5A4, and an outer cover 6B. Since bonding at the time of shaping the inner cover 5A4 into a cylindrical shape can be omitted, it is possible to easily manufacture the stent device 100H.

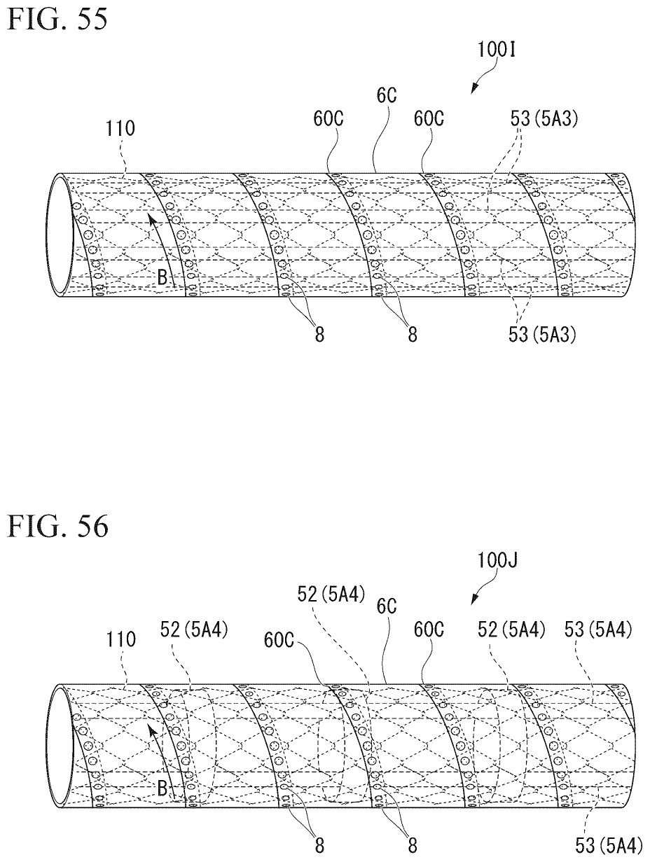

FIG. 55 is a diagram illustrating a stent device 100I which is an example of a combination.

The stent device 100I includes a stent 110, an inner cover 5A3, and an outer cover 6C. Since overlapping portions between the overlapping bonding portion 60C and the inner cover 5A3 are distributed, anisotropy of a reaction force to bending in the stent device 100I decreases.

FIG. 56 is a diagram illustrating a stent device 100J which is an example of a combination.

The stent device 100J includes a stent 110, an inner cover 5A4, and an outer cover 6C. Since overlapping portions between the overlapping bonding portion 60C and the inner cover 5A4 are distributed, anisotropy of a reaction force to bending in the stent device 100J decreases.

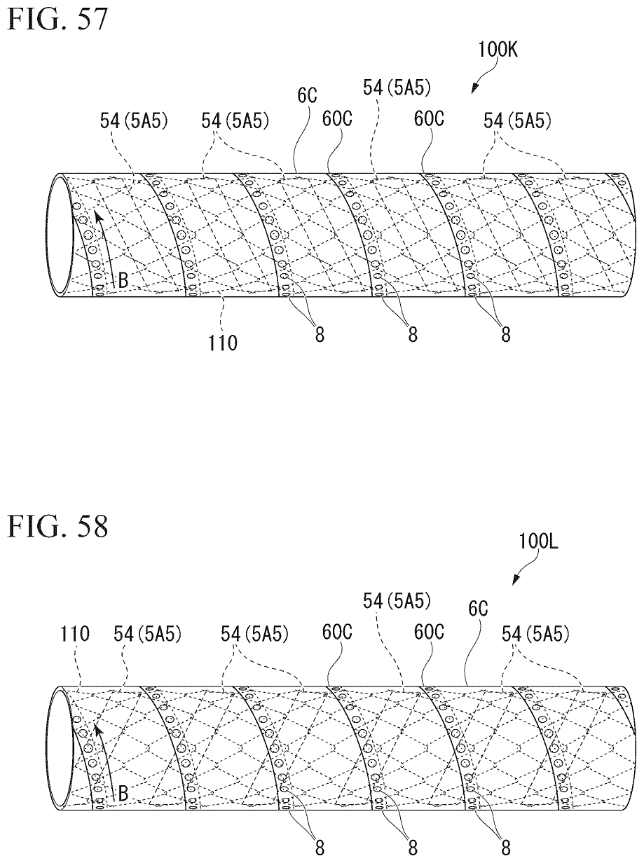

FIG. 57 is a diagram illustrating a stent device 100K which is an example of a combination.

The stent device 100K includes a stent 110, an inner cover 5A5, and an outer cover 6C. The spiral direction of the inner cover 5A5 wound in a spiral shape and the spiral direction of the outer cover 6C wound in a spiral shape are the same direction. In the stent device 100K, the overlapping bonding portion 60C and the inner cover 5A5 do not overlap. Accordingly, an amount of force required for releasing or re-capturing the stent device 100K decreases.

FIG. 58 is a diagram illustrating a stent device 100L which is an example of a combination.

The stent device 100L includes a stent 110, an inner cover 5A5, and an outer cover 6C. The spiral direction of the inner cover 5A5 wound in a spiral shape and the spiral direction of the outer cover 6C wound in a spiral shape are the reverse directions. It is possible to prevent a phenomenon in which the overlapping bonding portion 60C is open due to synergy of the overlapping portions between the overlapping bonding portion 60C and the inner cover 5A5.

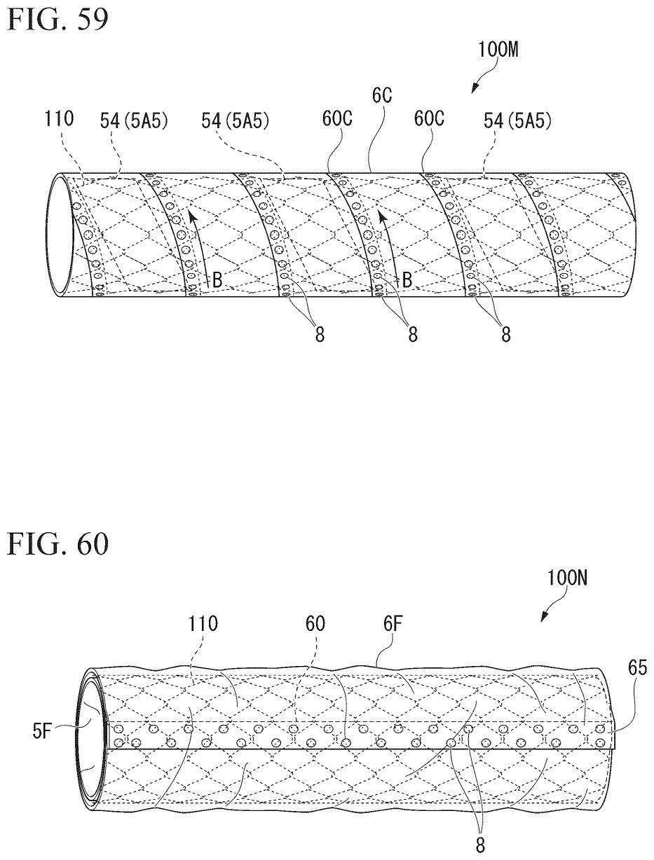

FIG. 59 is a diagram illustrating a stent device 100M which is an example of a combination.

The stent device 100M includes a stent 110, an inner cover 5A5, and an outer cover 6C. In the inner cover 5A5 of the stent device 100M, a width in a direction perpendicular to the spiral direction B is greater than that of the inner cover 5A5 of the stent device 100K. With the stent device 100M, it is possible to achieve an effect of prevention of breakage or separation in addition to an effect of prevention of migration. This is because the overlapping bonding portion 60C reinforces the inner cover 5A5 in a spiral shape.

FIG. 60 is a diagram illustrating a stent device 100N which is an example of a combination.

The stent device 100N includes an outer cover 6F which is formed in a cylindrical shape by the overlapping bonding portion 60 illustrated in FIG. 24. In the overlapping bonding portion 60, the bonding points 8 are alternately arranged in two lines (arranged in zigzag) in the longitudinal direction A.

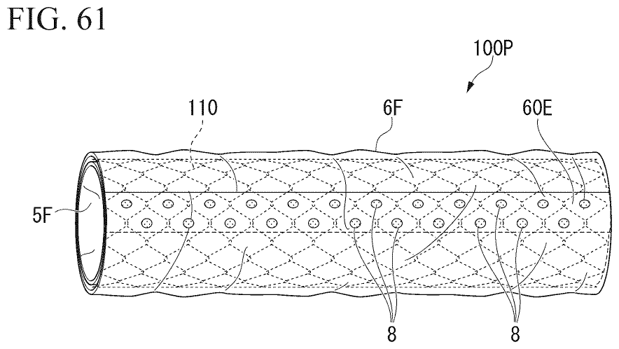

FIG. 61 is a diagram illustrating a stent device 100P which is an example of a combination.

The stent device 100P includes an outer cover 6F which is formed in a cylindrical shape by the overlapping bonding portion 60E illustrated in FIG. 36. In the overlapping bonding portion 60E, the bonding points 8 are alternately arranged in two lines (arranged in zigzag) in the longitudinal direction A. It is possible to prevent a phenomenon in which the overlapping bonding portion 60E is open at the time of infiltration of a tumor.

The present disclosure is applicable to a stent covered with a cover.

What is claimed is:

1. A stent device comprising:
a stent having a tubular shape, the stent comprised wires; and
a cover covering at least a part of the stent, the cover including an inner cover located inside of the stent and an outer cover located outside of the stent, and
wherein the inner cover and the outer cover are bonded in a cell area surrounded with the wires at a first end of the stent and a second end of the stent opposed to the first end in a longitudinal direction of the stent.

2. The stent device according to claim 1, wherein the inner cover includes a first tube located at the first end and a second tube located at the second end, and
wherein the first and second tube are bonded to the outer cover.

3. The stent device according to claim 2, wherein the first tube is spaced apart from the second tube,

4. The stent device according to claim 1, wherein the outer cover is bonded in an overlapping bonding portion in which a first end of the outer cover and a second end of the outer cover overlap each other.

5. The stent device according to claim 4, wherein the outer cover in the overlapping bonding portion is bonded at a plurality of bonding points.

6. The stent device according to claim 5, wherein the plurality of bonding points in the overlapping bonding portion are arranged at pitches of 2 mm to 4 mm.

7. The stent device according to claim 5, wherein the plurality of bonding points includes a first bonding point and a second bonding point next to the first bonding point, the wires are located between the first bonding point and the second bonding point.

8. The stent device according to claim 5, wherein the outer cover includes a first bonding point and a second bonding point next to the first bonding point, a non-bonded portion is located between the first bonding point and the second bonding point in the overlapping bonding portion, a length of the non-bonded portion is equal to or less than three times a length of the first bonding point.

9. The stent device according to claim 5, wherein the plurality of bonding points are arranged in the cell area when seen in a radial direction of the stent.

10. The stent device according to claim 5, wherein t the first end of the outer cover and the second end of the outer cover is in a circumferential direction of the stent.

11. The stent device according to claim 10, wherein the plurality of bonding points are arranged in two lines in the longitudinal direction of the stent.

12. The stent device according to claim 10, wherein the plurality of bonding points are alternately arranged in two lines in the longitudinal direction of the stent.

13. The stent device according to claim 5, wherein the overlapping bonding portion is formed in a spiral shape in the longitudinal direction of the stent.

14. The stent device according to claim 1, wherein the outer cover is located on an entire surface of the stent, and
wherein the inner cover is located on the entire surface of the stent.

15. The stent device according to claim 14, wherein the inner cover is bonded in an overlapping bonding portion in which a first end of the outer cover and a second end of the outer cover overlap each other.

16. The stent device according to claim 15, wherein the outer cover in the overlapping bonding portion is bonded at a plurality of bonding points.

17. The stent device according to claim 16, wherein the plurality of bonding points includes a first bonding point and a second bonding point next to the first bonding point, the wires are located between the first bonding point and the second bonding point.

18. The stent device according to claim 16, wherein the outer cover includes a first bonding point and a second bonding point next to the first bonding point, a non-bonded portion located between the first bonding point and the second bonding point in the overlapping bonding portion, a length of the non-bonded portion is equal to or less than three times a length of the first bonding point.

19. The stent device according to claim 14, wherein the outer cover has a tubular shape by wound in a spiral shape,
wherein the inner cover is wound in a spiral shape and is formed in a tubular shape, and
wherein a spiral direction of the inner cover and a spiral direction of the outer cover are parallel to each other.

20. The stent device according to claim 14, wherein the outer cover has a tubular shape by wound in a spiral shape,
wherein the inner cover is wound in a spiral shape and is formed in a tubular shape, and
wherein a spiral direction of the inner cover and a spiral direction of the outer cover are opposite to each other.
